SEQUENCE LISTING (2 sequences)

SEQ ID NO: 1 - DNA, 897 bp, Homo sapiens
agagactgcgagaaggaggtcccccacggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 2 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

(19) United States
(12) Patent Application Publication
(10) Pub. No.: US 2010/0212030 A1
(43) Pub. Date: Aug. 19, 2010

(54) USE OF N-TERMINAL AND C-TERMINAL PROTEOMICS TECHNOLOGY TO ENHANCE PROTEIN THERAPEUTICS AND DIAGNOSTICS

(75) Inventors: Sven Eyckerman, De Pinte (BE); Koen Kas, Schilde (BE)

Correspondence Address: KNOBBE MARTENS OLSON & BEAR LLP, 2040 MAIN STREET, FOURTEENTH FLOOR, IRVINE, CA, 92614, US

(73) Assignee: Pronota N.V., Zwijnaarde (BE)

(21) Appl. No.: 12/738,270

(22) PCT Filed: Oct. 17, 2008
(86) PCT No.: PCT/EP08/64039

§ 371 Date: Apr. 15, 2010

(30) Foreign Application Priority Data
Oct. 19, 2007 (EP) 07118859.3
Jan. 31, 2008 (EP) 08150911.9

(51) Int. Cl. C12P 21/00; C07K 1/00; C12Q 1/68

(52) U.S. Cl. 800/4; 530/402; 435/71.1; 435/6

(57) ABSTRACT

The present invention provides a novel method for stabilizing proteins, by first identifying the proteolytic sites using N- or C-terminal technology, followed by modification of said sites in order to create stabilized proteins, no longer subject to proteolytic cleavage. the method of the invention immediately provides the user with the exact amino acid position of the proteolytic cleavage site in the protein(s) of interest, even in a complex protein sample. This makes the specific modification of such a site much easier and increases the expectation of success as compared to the amount of effort needed, even in a complex protein sample.

### FIELD OF THE INVENTION

The invention relates to N-terminal and C-terminal technology which allows the identification and characterisation of novel (internal) N-termini or C-termini that are generated by proteolysis. This information can be used in modulating the sensitivity of proteins towards proteolytic cleavage and in the prognosis, diagnosis and/or treatment of diseases.

### BACKGROUND OF THE INVENTION

Numerous vaccines, therapeutics and diagnostic compositions comprise at least in part proteins or derivatives thereof. Such compositions are however prone to cleavage by proteases, e.g. residing in the blood stream or other tissues or systems in the subject to which the vaccine, therapeutic or diagnostic is administered. This proteolysis often results in degradation or inactivation of the active protein ingredient of the vaccine, therapeutic or diagnostic composition. In order to stabilize such proteins, the present invention provides 1) means and methods for unambiguously identifying proteolytic cleavage sites in a certain protein and 2) means and methods of modifying said identified sites in order to alter the proteolytic cleavage in said protein and thereby possibly modifying the half-life and/or activity of said protein, especially when administered or produced in an in vivo environment.

Several strategies to increase the stability of proteins have been developed in the prior art. Examples are the introduction of disulfide bonds in proteins as is described in US patent application US 2005/0123530A1 by Marshall et al., thereby strengthening the three-dimensional structure of the protein. In an alternative approach, combinatorial libraries of consensus mutations in combination with screening methods for stabilized mutants are used as is explained in international patent application WO 2005/040344. Another example is U.S. Pat. No. 6,385,546, describing a method of identifying and changing amino acid residues that affect the stability of a protein and thereby to “adjust” the stability of a protein under particular conditions, e.g., to function at lower or higher temperatures. The residues modified in the method are chosen such that they are not in or interact with an active site or binding site of the protein, and therefore the mode of action or interaction of the protein on substrates remains unchanged. In alternative embodiments, proteins are stored in specific buffers that stabilise the protein. Examples are glycerine buffers, HEPES, TES and TRICINE. In further examples, the protein preparation is lyophilized or freeze dried for storage.

International patent application WO 93/11254 discloses a method to prevent proteolytic cleavage of a protein, wherein one or more protease labile amino acid segments are substituted by protease non-labile amino acid segment(s). The stabilized protein may be produced by recombinant DNA techniques, introducing randomized mutations in the protein and subsequently analysing the proteolytic cleavage of the protein variant. The method however does not correctly identify the proteolytic cleavage sites, but uses a random mutation procedure.

There is therefore a clear need for improved techniques for stabilizing proteins for use in vaccines and therapeutics or diagnostics which are constituted out of protein compounds.

The present invention provides a novel method for stabilizing proteins, by first identifying the proteolytic sites using N-terminal or C-terminal technology, followed by modification of said identified proteolytic cleavage sites in order to create proteins that have an altered sensitivity towards proteolytic cleavage.

### SUMMARY OF THE INVENTION

The invention provides means and methods for identifying proteolytic cleavage sites in one or more protein(s) in a protein mixture and subsequently modifying said identified proteolytic cleavage site(s) in order to alter the sensitivity of said protein(s) towards proteolytic cleavage. This may result either in an increased half-life of said protein(s), especially in an in-vivo environment, or in an altered protein activity since it is well known that proteolytic cleavage can both lead to activation or inactivation or increase or decrease of activation level of proteins. The advantage of such a modified protein is 1) to reduce dosing of these proteins when administrated in a subject, as a pharmaceutical, a diagnostic or as a vaccine and 2) to improve methods of in vivo production of such proteins in e.g. a transgenic animal or in a microbacterial system. The advantage of increasing the activity of a protein can be e.g. be to restore natural functionality of said protein. Alternatively, it can be beneficial to decrease the activity of a certain protein in certain disease conditions where said protein is hyperactive. The person skilled in the art would be aware of other conditions in which (partial) activation or (partial) inactivation of a certain protein involved in said disease or condition, would be beneficial to a patient.

In one embodiment, the invention provides for a method for increasing the half-life and/or modulating the activity of one or more protein(s) comprising 1) identifying the novel internal proteolytic cleavage site(s) in said one or more protein(s) using N-terminal or C-terminal technology, 2) modifying said identified proteolytic cleavage site(s) in said one or more protein(s) such that the sensitivity of said one or more protein(s) towards proteolytic cleavage at said identified site(s) is modulated. In a preferred embodiment, the half-life and/or activity of said protein in an in-vivo environment is modified.

In an alternative embodiment, the invention provides for an improved method for the production of one or more protein(s) in a protein mixture comprising the steps of a) identifying the proteolytic cleavage site(s) that lead to protein cleavage during the production process in said protein using N-terminal or C-terminal technology, b) modifying said identified proteolytic cleavage site(s) in the protein(s) such that the sensitivity of said protein towards proteolytic cleavage at said site(s) is altered, thereby altering its stability in the in vivo production process e.g. in a transgenic animal or in a microbacterial system.

In a further embodiment, the invention provides a method of detecting naturally occurring SNPs that are connected to a disease or disorder related to proteolytic cleavage of a protein comprising the steps of:

a) identifying the proteolytic cleavage site(s) that lead to protein cleavage during the production process in said protein using N-terminal or C-terminal technology,
b) searching an SNP database for mutations in the isolated ApoA1 protein that correspond to the newly identified proteolytic cleavage site in step a).

Alternatively, the invention provides a method for diagnosing a disease or disorder related to proteolytic cleavage of a protein comprising the steps of detecting one or more SNPs identified by the method above, in a sample of said patient. Said disease or disorder can e.g. be linked to increased or excessive proteolytic cleavage of a protein or to lack of, decrease in, or insufficiency in proteolytic cleavage of said protein.

In some embodiments, said one or more protein(s) is or forms part of a protein-based medicament, a pharmaceutical composition, a vaccine, or a diagnostic composition.

The invention thus further provides for a vaccine or a diagnostic composition with an improved half-life or shelf-life, due to the increase in stability or half-life of the protein which it comprises as the active ingredient.

In some embodiments, said one or more protein(s) are produced synthetically or recombinantly using methods known in the art.

In some embodiments, the modification of the identified proteolytic cleavage site(s) is done by introduction of one or more point mutation(s) in the nucleic acid coding sequence of the protein at a position overlapping with and/or surrounding said identified proteolytic cleavage site(s), thereby altering the amino acid sequence of the protein(s) and subsequently blocking, inhibiting or reducing proteolytic cleavage.

In some embodiments, the modification of the identified proteolytic cleavage site(s) is done by chemical modification of one or more side chains of the amino acid residues overlapping with and/or surrounding said identified proteolytic cleavage site(s), subsequently blocking, inhibiting or reducing proteolytic cleavage.

In some embodiments, the modification of the identified proteolytic cleavage site(s) is done by introducing one or more non-natural amino acids encoded by specific non-natural codons which are introduced in the coding sequence of the target protein or by replacing existing amino acids in the sequence of the protein by non-natural amino acids or by deleting one or more amino acids of the protein sequence or by substituting one or more amino acids in the protein sequence for another amino acid, e.g. by substituting the amino acids with structurally related amino acids (e.g. basic, acidic, uncharged or non-polar amino acids are substituted by other respectively basic, acidic, uncharged or non-polar amino acids).

In some embodiments, the proteolytic cleavage at the identified proteolytic cleavage site(s) is modulated by binding an affinity ligand or a binding molecule to the proteolytic cleavage site(s) in the target protein(s) or to its protease(s), preventing or reducing the interaction between the target protein and its protease.

In some embodiments, the proteolytic cleavage at the identified proteolytic cleavage site(s) is reduced by inhibiting the protease(s) responsible for cleaving the protein(s) of interest in the protein mixture using one or more inhibiting agent(s). The aminoacid context of the cleavage site may help in identification of the protease as some of these proteases have well-defined substrate specificities.

In some embodiments , the method for identification of proteolytic cleavage site(s) in one or more protein(s) present in a protein mixture comprises the steps of: a) optionally selecting a protein of interest from the protein mixture using a specific binding molecule or a combination of several specific binding molecules, b) modifying or labelling all true and/or novel internal N-termini or C-termini of the protein(s) in the protein mixture, c) cleaving or hydrolysing the proteins in the protein mixture into peptides with e.g. trypsin, chymotrypsin and the like, d) optionally separating the modified or labelled N-terminal or respective C-terminal peptides from the non-modified or non-labelled peptides in the protein mixture, e) analyzing only the modified or labelled N-terminal or respective C-terminal peptides from the mixture using mass-spectrometric methods, and f) identifying all internal proteolytic cleavage site(s) of said one or more protein(s) in said protein mixture.

In some embodiments, the N-terminal or C-terminal modification step b) is done by blocking the true and/or novel internal N-termini or respective C-termini with a specific agent and the optional separation step d) is done by using aminopeptidase or respective carboxypeptidase degrading only the non-protected peptides in the protein mixture into single amino acid residues.

In some embodiments, the N-terminal or C-terminal labelling step b) is done by addition of a capturing-molecule on the true and/or novel internal N-termini or respective C-termini of the protein(s) and wherein the optional separation step d) is done by capturing only the labelled peptides on a solid support or by capturing only the non-labelled peptides on a solid support.

In some embodiments, said capturing-molecule is selected from the group of beads, glass beads, controlled-pore silicate glass beads such as biotin, PITC or DITC, an organic cyclic compound such as a crown ether or a derivative thereof, MIPs, DARPins, a fluorous or cis-diol moiety or any other molecule designed to selectively bind to the primary amine groups of the N-termini or any other molecule that binds selectively to the novel C-termini of proteins, and wherein the separation step is done by column purification, affinity capture, filtration, centrifugation, magnetic capture, matrix capturing or the like.

In some embodiments, the protein mixture is derived from a complex body sample selected form the group of blood, plasma, serum, urine, faeces, saliva, cerebrospinal fluid, nipple aspirate, ductal lavage, sweat or perspiration, tumor exudates, joint fluid (e.g. synovial fluid), inflammation fluid, tears, semen, vaginal secretions and tissue biopsies and wherein the protein mixture comprises one or more proteins, wherein said proteins can be present one or more isoforms.

In some embodiments, the analysis of the true and/or novel internal N-terminal or C-terminal peptides is done by using electrospray ionization mass spectrometry, ion trap mass spectrometry, hybrid ion trap mass spectrometry coupled to quadrupole, time-of-flight mass spectrometry, or a reversed phase-high performance liquid chromatography system connected to a nanospray ionization hybrid ion trap-fourier transform mass spectrometer.

These and further aspects and preferred embodiments of the invention are described in the following sections and in the appended claims.

### BRIEF DESCRIPTION OF THE DRAWINGS

FIG. 1:

The number of peptides identified by N-terminal COFRADIC and their corresponding starting position in the pre-pro-ApoA1 protein. The sample used is serum obtained from a healthy volunteer. The number of peptide identifications gives a rough estimate on the extent of cleavage at the particular position, showing thus extensive cleavage in the pre-pro-ApoA1 protein after R184. The protein and its processed form are shown schematically below the graph.

FIG. 2:

FIG. 2a shows the complete nucleotide sequence of the mRNA encoding the pre-pro-ApoA1 protein, available as NM_{—}000039 in Genbank.

FIG. 2b shows the amino acid sequence of the pre-pro ApoA1 protein, available as NP_{—}000030 in Genbank and indicates the starting points of the pre-form (starting at AA residue 1, pro-form (starting at AA residue 19) and the mature ApoA1 protein (starting at AA residue 25).

### DETAILED DESCRIPTION OF THE INVENTION

The inventors used N-terminal or C-terminal peptide-identification technologies to identify novel internal N-termini or respective C-termini that are generated by proteolysis. Protein cleavage is an important step in degradation, activation and/or processing of proteins and appears to be an essential process in serum or plasma homeostasis. Cleavage of target protein substrates can directly or indirectly affect the activity or function of numerous proteins, enzymes and receptors, and other proteins within a biological pathway. This can lead to a cascade of events that may trigger intracellular signaling or may lead to changes in various cell activities, including, for example, cell spreading, migration, cell-cell adhesion, ectodomain shedding and cell death. In addition, the coagulation and complement pathways are well-characterized examples of highly-regulated proteolytic cascades that operate in blood.

By analyzing serum samples from healthy human subjects, the inventors discovered that proteolysis leads to the formation of an unexpectedly high amount of protein fragments derived from naturally synthesised proteins. The invention relates to methods to reduce complexity of the protein mixture and to the development of a method to identify internal proteolytic cleavage sites in proteins using an N-terminal or C-terminal technology.

The method comprises the selective isolation of peptides derived from the N-terminal or C-terminal ends of proteins in a complex protein sample after proteolysis, followed by analysis and identification of the peptides in the mixture. Besides identifying the true N-terminus or C-terminus of the proteins in the mixture e.g. after maturation and cleavage of pre and pro domains, the N-terminal or respective C-terminal peptide-identification technology of the invention also allows the characterisation of novel internal N-termini or respective C-termini that are generated by proteolysis.

The method of the invention is advantageous over the methods currently known in the art in that it combines N-terminal or C-terminal technology for unambiguously and directly identifying internal proteolytic cleavage sites with a method of modifying such sites in order to create more stable proteins. Whereas the methods known in the art have to make use of cumbersome methods such as random mutagenesis, epitope tagging, amino acid modeling or 3-dimensional structural analysis in order to predict the possible site(s) of proteolysis, the method of the invention immediately provides the user with the exact amino acid position of the proteolytic cleavage site(s) in the protein(s) of interest, even in a complex protein sample. This makes the specific modification of such a site much easier and increases the expectation of success as compared to the amount of effort needed, even in a complex protein sample.

### N-Terminal and C-Terminal Technologies

The invention uses the N-terminal or C-terminal technologies to identify endogenous proteolytic events and cleavage sites in proteins. Any N-terminal or C-terminal technology can be used in the method of the invention. By way of non-limiting example, some set-ups of N-terminal and C-terminal technologies are explained below.

In a general exemplary embodiment, the method for identification of proteolytic cleavage sites in a protein comprises the steps of a) optionally selecting and/or enriching one or more protein(s) of interest using specific binding molecules or a mixture of binding molecules specifically recognizing said protein(s), in order to reduce protein complexity of the sample, b) blocking or labelling all genuine N-termini or C-termini (i.e. all N-termini or C-termini generated by proteolytic cleavage) of the proteins in a complex sample, c) cleaving or hydrolysing the extracted proteins in the mixture into peptides with e.g. trypsin, chymotrypsin and the like, d) optionally separating the modified N-terminal or respective C-terminal peptides from the non-modified peptides, e) analyzing the modified or captured N-terminal or respective C-terminal peptides obtained in step d) using mass-spectrometric methods, and f) identifying all novel internal proteolytic cleavage site(s) in said protein.

In one such embodiment, the genuine N-terminal or C-terminal peptides are labelled by addition of a capturing-molecule on the genuine N-termini or respective C-termini of the protein, followed by fragmentation of the proteins in the protein mixture into peptides, after which a purification or selection step is performed capturing all capturing-molecule-linked N-terminal or respective C-terminal peptides on a solid phase and discarding the unwanted internal peptides.

In another embodiment, the true N-terminal or C-terminal peptides are blocked, followed by fragmentation of the proteins in the protein mixture into peptides, after which the non-blocked internal peptides are optionally labelled and subsequently captured on e.g. a solid phase such as a matrix or a column, or through filtration or centrifugation steps of bead-labelled internal peptides, leaving over the wanted N-terminal or C-terminal peptides for analysis through MS.

The separation of the genuine N- or C-termini can thus be done by specifically labelling or tagging said genuine N- or C-termini, followed either by capturing only the non-labelled internal peptides, or by capturing only the labelled genuine N- or C-termini on a solid phase.

Alternatively, the separation of the genuine N- or C-termini can be done by selectively labelling or tagging the internal peptides only, followed either by capturing only the labelled internal peptides, or by capturing only the non-labelled genuine N- or C-termini on a solid phase.

Using techniques well known in the art according to the tag or label, the labelled peptides can be separated from the non-labelled peptides, subsequently resulting in a pool of N-terminal or C-terminal peptides from the complex sample.

In a preferred embodiment, said labelling or tagging molecules are selected from the group of beads, glass beads, controlled-pore silicate glass beads such as PITC or DITC, an organic cyclic compound such as a crown ether or a derivative thereof, an antibody (e.g. a monoclonal or polyclonal antibody or fragments thereof), nanobody, affybody, aptamer, Molecular Imprinting Polymers (MIPs), DARPins, a fluorous or cis-diol moiety or any other molecule designed to selectively bind to the primary amine groups of the N-termini or any other molecule that binds selectively to the novel C-termini of proteins. Using techniques well known in the art according to the label, the labelled peptides can be separated from the non-labelled peptides, resulting in a pool of N-terminal or C-terminal peptides from the complex sample.

In another embodiment, the invention uses a method for isolating N-terminal peptides from a protein or mixture of proteins, comprising:

(a) protecting the N-terminal amino acid in the protein or in proteins of the protein mixture by a pre-treatment,
(b) fragmenting the protein or the protein mixture from (a) to obtain a protein peptide mixture, and
(c) reacting the protein peptide mixture from (b) with an aminopeptidase, whereby said N-terminal peptides are isolated.

In another aspect the invention uses a method for isolating C-terminal peptides from a protein or mixture of proteins, comprising:

(a) protecting the C-terminal amino acid in the protein or in proteins of the protein mixture by a pre-treatment,
(b) fragmenting the protein or the protein mixture from (a) to obtain a protein peptide mixture, and
(c) reacting the protein peptide mixture from (b) with a carboxypeptidase, whereby said C-terminal peptides are isolated.

### Pre-Treatments in the Amino- or Carboxypeptidase Mediated Technology

As noted, in the present methods the protein or protein mixture is subjected to a pre-treatment, such as to desirably protect the N-terminal amino acid or the C-terminal amino acid in the protein or in proteins of the protein mixture. This desirably blocks said N-terminal amino acid or said C-terminal amino acid, such as to prevent their cleaving-off by the action of aminopeptidase or carboxypeptidase, respectively.

Suitable blocking reagents, as well as methods and conditions for attaching and detaching protecting groups will be clear to the skilled person and are generally described in standard handbooks of organic chemistry, such as “Protecting Groups”, P. Kocienski, Thieme Medical Publishers, 2000; Greene and Wuts, “Protective groups in organic synthesis”, 3rd edition, Wiley and Sons, 1999; incorporated herein by reference in their entirety.

Preferably, protection of the N-terminal amino acid can be achieved by suitably modifying the α-NH₂ group of said N-terminal amino acid. For example, said α-NH₂ group can be modified using reagents capable of selectively reacting with primary amino groups (“primary amino” alone or in combination refers to a group of formula —NH₂, optionally in any dissociation or protonation state such as —NH₃⁺) and presenting a non-reactive substituent for subsequent conditions. A blocking reagent may be generally substituted once or twice on each so-modified primary amine (i.e., —NH₂ gives —NHZ or —NZ₂, where Z is the substituent introduced by said blocking reagent).

In a non-limiting and preferred example, primary amines may be protected by acylation, e.g., acetylation, using reagents known per se, such as, e.g., using acetyl N-hydroxysulfosuccinimide, 2,4,6-trinitrobenzene sulfonic acid (TNBS), formaldehyde or any other group for reductive amination, ICPL (Serva) and ITRAQ (applied Biosystems) reagents. Other suitable primary amino-modifying reagents have been extensively described in the art, for example, in Regnier et al. 2006 (Proteomics 6: 3968-3979). During modification of —NH₂ groups with acyl such as acetyl, the acyl moiety may be occasionally also introduced on the —OH group of Ser, Thr and/or Tyr. Such ester bonds are preferably subsequently broken by alkali hydrolysis at conditions that do not affect the acylation of the —NH₂ groups.

Preferably, protection of the C-terminal amino acid can be achieved by suitably modifying the α-COOH group of said C-terminal amino acid. For example, said α-COOH group can be modified using reagents capable of selectively reacting with carboxyl groups (“carboxyl” alone or in combination refers to a group of formula —COOH, optionally in any dissociation or protonation state such as —COO′) and presenting a non-reactive substituent for subsequent conditions.

In non-limiting and preferred examples, carboxyl groups may be protected by esterification to methyl esters, t-Butyl esters, benzyl esters, S-t-Butyl esters, or by conversion to 2-alkyl-1,3-oxazoline, to 5,6-Dihydrophenanthridinamide or to hydrazide using reagents known per se (see, e.g., Greene and Wuts 1999, supra).

Further advantageous pre-treatments of the protein mixture or protein peptide mixture may be included. For instance, Cys —SH groups in the protein, protein mixture or protein peptide mixture can be protected to avoid their reactivity, in particular oxidation, throughout the methods. Typically, the sample is first treated with a reducing agent known per se, such as, e.g., β-mercaptoethanol, dithiothreitol (DTT), dithioerythritol (DTE) or a suitable trialkylphosphine inter alia tris(2-carboxyethyl)phosphine (TCEP), to quantitatively reduce any oxidised —SH groups, e.g., disulphide bridges. The —SH groups are subsequently protected with a blocking reagent that reacts selectively with Cys side chains and presents a non-reactive substituent for subsequent conditions. By means of example and not limitation, —SH groups may be converted to acetamide derivatives by treatment with iodoacetamide in denaturing buffers (e.g., guanidium- or urea-containing buffers). Other blocking reagents, such as N-substituted maleimides (e.g., N-ethylmaleimide), acrylamide, N-substituted acrylamide or 2-vinylpyridine, may alternatively be used.

Pre-treatments may be applied simultaneously or sequentially in any suitable order. After and during pre-treatment, the sample may be optionally be purified using known techniques, such as solvent evaporation, washing, filtration, chromatographic techniques, etc.

### Fragmentation in the Amino- or Carboxypeptidase Mediated Technology

A protein peptide mixture may be obtained by fragmentation of a protein or mixture of proteins, such as, e.g., by fragmentation of all or a fraction of proteins present in and/or isolated from a biological sample after the sample has been removed from biological source.

The term “fragmentation” as used herein in relation to a protein refers to cleavage, preferably enzymatic or chemical cleavage, of one or more peptide bonds within said protein or within any one or more of its polypeptide chains. Fragmentation of protein mixture denotes fragmentation of proteins constituting said protein mixture. Advantageously, proteins or protein mixtures may be fragmented so as to yield protein peptide mixtures having the preferred average or median chain lengths as detailed above.

When a protein or a polypeptide chain is cleaved at least at one peptide bond, such fragmentation generates a peptide that comprises the N-terminal end of said protein or polypeptide chain (“N-terminal peptide”) and a peptide that comprises the C-terminal end of said protein or polypeptide chain (“C-terminal peptide”). Where the protein or polypeptide chain is cleaved at two or more of its peptide bonds, such fragmentation additionally produces one or more peptides derived from the portion of the protein or polypeptide chain interposed between the parts corresponding to the N- and C-terminal peptides (“internal peptides”).

To ensure optimal characterisation of N-terminal or C-terminal peptides, it is desirable that fragmentation of individual molecules of a given protein occurs at the same peptide bond in substantially all individual molecules of said protein.

This can be advantageously achieved when the protein or protein mixture is fragmented preferentially at peptide bonds N-terminally or C-terminally adjacent to one or more specific amino acid residue types (denoted as X¹ . . . Xⁿ). The term “fragmented preferentially at” means that the fragmentation occurs substantially only at the recited peptide bond(s). Preferably, less than 10% of peptide bonds other than the recited ones would be cleaved, e.g., <7%, more preferably <5%, e.g., <4%, 3% or <2%, most preferably <1%, e.g., <0.5%, <0.1%, or <0.01%.

Preferably, a protein or protein mixture will be fragmented at substantially all recited peptide bonds. Hence, the fragmentation would occur substantially quantitatively at peptide bonds N-terminally or C-terminally adjacent to residues of the one or more types X¹ . . . Xⁿ.

To achieve a protein peptide mixture displaying preferred average and/or median peptide lengths, the protein or protein mixture may be advantageously fragmented adjacent to a relatively small number of amino acid residue types X¹ . . . Xⁿ, such as at peptide bonds adjacent to 5 or less amino acid residue types (i.e., n≦5), more preferably r^{˜}14, even more preferably n≦3, still more preferably n≦2, or preferably at peptide bonds adjacent to only 1 amino acid residue type (i.e., n=1).

The one or more specific amino acid residue types X¹ . . . Xⁿ adjacent to which fragmentation is contemplated herein may be selected from any amino acid residues, including but not limited to amino acids found in naturally occurring proteins, amino acids carrying a co- or post-translational modification, amino acids including a non-natural isotope, or amino acids further chemically and/or enzymatically altered prior to the fragmentation, etc.

A suitable frequency of cleavage may be preferably achieved when the fragmentation takes place adjacent to one or more of the 20 common amino acid residue types found in natural proteins and/or adjacent to one or more of residue types obtained from any of the 20 common amino acid residue types by suitable modification of the starting proteins. Accordingly, in a preferred embodiment, the protein or mixture of proteins is fragmented preferentially at peptide bonds adjacent to one or more amino acid residue types X¹ . . . Xⁿ chosen from the group consisting of: Gly, Pro, Ala, Val, Leu, Ile, Met, Cys, Phe, Tyr, Trp, His, Lys, Arg, Gln, Asn, Glu, Asp, Ser and Thr; optionally including a co- or post-translational modification, a chemical and/or enzymatic alteration prior to the fragmentation, or including a non-natural isotope, etc.

Fragmentation may be effected by suitable physical, chemical and/or enzymatic agents, more preferably chemical and/or enzymatic agents, even more preferably enzymatic agents, e.g., proteinases, preferably endoproteinases. Preferably, the fragmentation may be achieved by one or more, preferably one, endoproteinase, i.e., a protease cleaving internally within a protein or polypeptide chain (i.e., endoproteolytic cleavage or fragmentation). A non-limiting list of suitable endoproteinases includes serine proteinases (EC 3.4.21), threonine proteinases (EC 3.4.25), cysteine proteinases (EC 3.4.22), aspartic acid proteinases (EC 3.4.23), metalloproteinases (EC 3.4.24) and glutamic acid proteinases.

By means of example not limitation, protein fragmentation may be achieved using trypsin, chymotrypsin, elastase, Lysobacter enzymogenes endoproteinase Lys-C, Staphylococcus aureus endoproteinase Glu-C (endopeptidase V8) or Clostridium histolyticum endoproteinase Arg-C (clostripain). The invention encompasses the use of any further known or yet to be identified enzymes; a skilled person can choose suitable protease(s) on the basis of their cleavage specificity and the frequency of occurrence of the amino acid(s) adjacent to which fragmentation is induced, to achieve desired protein peptide mixtures.

In a preferred embodiment, the fragmentation may be effected by endopeptidases of the trypsin type (EC 3.4.21.4), preferably trypsin, such as, without limitation, preparations of trypsin from bovine pancreas, human pancreas, porcine pancreas, recombinant trypsin, Lys-acetylated trypsin, etc. Trypsin is particularly useful in proteomics applications, inter alfa due to high specificity (C-terminally adjacent to Arg and Lys except where the next residue is Pro) and efficiency of cleavage. The invention also contemplates the use of any trypsin-like protease, i.e., with a similar specificity to that of trypsin.

It has been suggested that some aminopeptidases may cleave-off N-terminal proline with reduced efficiency. To avoid this, fragmentation of proteins to protein peptide mixtures may be advantageously performed using a prolyl endopeptidase (EC 3.4.21.26), i.e., endopeptidase that specifically cleaves C-terminally to Pro, such as by example but without limitation the recombinant Pro-C endopeptidase available from Fluka (Cat. No. 45167). Hereby, Pro would become the ultimate residue of unwanted peptides, which would therefore be completely hydrolysed by aminopeptidase.

In other embodiments, chemical reagents may be used. By means of example and not limitation, CNBr can fragment proteins at Met; BNPS-skatole can fragment at Trp.

The conditions for treatment, e.g., protein concentration, enzyme or chemical reagent concentration, pH, buffer, temperature, time, can be determined by the skilled person depending on the enzyme or chemical reagent employed.

### Exopeptidases

Methods of the invention employ exopeptidases, namely aminopeptidases or carboxypeptidases, to hydrolyse unwanted unprotected peptides, thus leaving behind and enriching for desired protected N-terminal or C-terminal peptides, respectively.

As used herein, the term “exopeptidase” refers to a hydrolase enzyme which hydrolyses the peptide bonds adjacent to terminal amino acids of a peptide or protein, thereby removing said terminal amino acids from said peptide or protein.

The term “aminopeptidase” refers to an exopeptidase which hydrolyses the peptide bond adjacent to the N-terminal amino acid of a peptide or protein, thereby releasing said N-terminal amino acid from said peptide or protein.

Exemplary but non-limiting of aminopeptidases are grouped under EC classification numbers EC 3.4.11.1 to EC 3.4.11.23. Engineered aminopeptidases with optimal or evolved enzymatic characteristics for removal of aminoacids are also covered by this term. The term “carboxypeptidase” refers to an exopeptidase which hydrolyses the peptide bond adjacent to the C-terminal amino acid of a peptide or protein, thereby releasing said C-terminal amino acid from said peptide or protein. Exemplary but non-limiting carboxypeptidases are grouped under EC classification numbers EC 3.4.16 (serine-type carboxypeptidases), EC 3.4.17 (metallocarboxypeptidases) and EC 3.4.18 (cysteine-type carboxypeptidases). Engineered carboxypeptidases with optimal or evolved enzymatic characteristics for removal of aminoacids are also covered by this term. In an embodiment, an aminopeptidase or carboxypeptidase may display substantially no preference or specificity for the type of amino acid that it cleaves-off, such that it would successively remove all amino acid types from a peptide's N-terminus or C-terminus, respectively, thereby completely hydrolysing the peptide. Non-limiting examples of non-specific aminopeptidases include inter alia aminopeptidase I from Streptomyces griseus (Spungin & Blumberg 1989, Eur J Biochem 183: 47; EC 3.4.11.22, #A9934 Sigma Aldrich), Microsomal aminopeptidase M from Sus scrofa (EC 3.4.11.2, #L5006 Sigma Aldrich), Aeromonas proteolytica aminopeptidase (EC 3.4.11.10, #A8200 Sigma Aldrich), and porcine leucine aminopeptidase (EC 3.4.11.1). Non-limiting examples of non-specific carboxypeptidases include inter alia carboxypeptidase C and Y (EC 3.4.16.5), and Carboxypeptidase P. In another embodiment, the methods may employ aminopeptidases or carboxypeptidases that display preference or specificity for cleaving-off one or more particular amino acid types. In this embodiment, to achieve successive release of all amino acid types from a peptide's N-terminus or C-terminus, combinations of two or more aminopeptidases with complementary specificities or of two or more carboxypeptidases with complementary specificities, respectively, may be used. The combination of prolyl aminopeptidase (EC 3.4.11.5 removing N-terminal prolines) with Aminopeptidase M (EC 3.4.11.2) can compensate for the delayed activity on N-terminal prolines. Aminopeptidases or carboxypeptidases for use herein may be isolated as known in the art from a variety of respective sources, and also include any recombinantly produced forms thereof. The conditions for peptide hydrolysis, e.g., peptide concentration, exopeptidase concentration, pH, buffer, temperature, time, post-reaction inactivation, etc., can be determined by the skilled person depending on the enzyme employed.

### Separation of N-Terminal or C-Terminal Peptides in the Amino- or Carboxypeptidase Mediated Technology

Depending on parameters such as the complexity of the protein sample, the N-terminal or C-terminal peptides isolated as above can be directly subjected to methods for peptide identification, or may be further resolved (fractionated) using a single- or multi-dimensional separation process prior to such identification. In a “single-dimensional” separation process a sample of analytes (peptides) is subjected to a single separation step which resolves analytes on the basis of one or more, such as one, physical and/or chemical property. In a “multi-dimensional” separation process a sample of analytes is subjected to a sequence of two or more separation steps (“dimensions”), each of which acts upon all or a part of analytes separated in a previous separation step, wherein any two analytes resolved in a given separation step remain resolved in subsequent separation steps, and wherein the distinct separation steps resolve analytes on the basis of different physical and/or chemical properties. Preferably, the distinct separation steps are orthogonal, such that peptides not resolved (i.e., recovered in same fraction) in one step will be resolved in another step. Typically, to realise a multidimensional separation, any or all fractions from a given separation step are each individually resolved in a subsequent separation step. Analytical separation methods that can fractionate peptides on the basis of one or more physical and/or chemical properties are well-known in the art. For example, electrophoresis applications exist to resolve peptides on the basis of net charge, EPM or pI, including inter alia gel electrophoresis such as capillary gel electrophoresis (CGE), capillary zone electrophoresis (CZE), free flow electrophoresis (FFE), isoelectric focusing (IEF) including capillary isoelectric focusing (CIEF), isotachophoresis (ITP), capillary electrochromatography (CEC), and the like. For example, size exclusion chromatography (SEC) including gel filtration chromatography or gel permeation chromatography may be applied to resolve peptides based on molecular size. In a particularly preferred example, peptides may be resolved by chromatography, preferably 1D- or 2D-chromatography. The term “chromatography” includes methods for separating chemical substances, referred to as such and vastly available in the art. In a preferred approach, chromatography refers to a process in which a mixture of chemical substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is also widely applicable for the separation of chemical compounds of biological origin, such as, e.g., amino acids, proteins, fragments of proteins or peptides, etc. Exemplary types of chromatography useful herein include, without limitation, high-performance liquid chromatography (HPLC), normal phase HPLC (NP-HPLC), reversed phase HPLC(RP-HPLC), ion exchange chromatography, such as cation or anion exchange chromatography, hydrophilic interaction chromatography (HILIC), hydrophobic interaction chromatography (HIC), affinity chromatography such as immuno-affinity and immobilised metal affinity chromatography. While particulars of these chromatography types are well known in the art, for further guidance see, e.g., Meyer M., 1998, ISBN: 047198373X and Cappiello et al. 2001 (Mass Spectrom Rev 20: 88-104), incorporated herein by reference. Preferably, the chromatography may employ liquid mobile phase (i.e., liquid chromatography). Also preferably, the chromatography may be columnar, i.e., wherein the stationary phase is deposited or packed in a column. In yet further preferred embodiment, the chromatography is HPLC, such as preferably RP-HPLC. Columns and conditions for performing HPLC separations including RP-HPLC are generally known to the skilled person, and described in, e.g., Practical HPLC Methodology and Applications, Bidlingmeyer, B. A., John Wiley & Sons Inc., 1993.

### Identification and Quantification of Peptides and Proteins

The N-terminal or C-terminal peptides isolated and optionally fractionated using any of the methods described above, represent those peptides starting (N-terminal) or ending (C-terminal) at the internal proteolytic cleavage sites of the protein(s) of interest in the protein mixture and can thus be used to identify the corresponding proteolytic cleavage site(s) in one or more protein(s) of interest in a complex starting sample.

In a preferred approach, further separation, analysis and/or identification of the peptides may be performed using a mass spectrometer. Otherwise, said peptides may be analysed and/or identified using other methods such as, e.g., activity measurement in assays, analysis with specific antibodies, Edman sequencing, etc. In an embodiment, N-terminal or C-terminal peptides released from the isolation or separation process can be directly (on-line) fed to an analyser (e.g., on-line LC/MS/MS). Otherwise, the peptides resolved by the separation process may be collected in fractions which, optionally following additional manipulation (e.g., concentration and/or spotting onto a MALDI-target; or advantageously, mixing with matrix in a microtee prior to deposition on MALDI targets, thereby eliminating the need for concentration and manual spotting; etc.), can be fed to an analyser. Preferably, the peptides are analysed and identified using mass spectrometry (MS), preferably high-throughput MS techniques known per se that can obtain precise information on the mass and preferably also on (partial) amino acid sequence of the peptides (e.g., in tandem mass spectrometry, MS/MS; or in post source decay TOF MS). Such information can be used in database searching to trace the peptides back to their parent proteins. MS arrangements and instruments appropriate for peptide analysis are commonly known and may include, without limitation, matrix-assisted laser desorption/ionisation time-of-flight (MALDI-TOF) MS systems; MALDI-TOF post-source-decay (PSD) systems; MALDI-TOF/TOF systems; electrospray ionisation (ESI) 3D or linear (2D) ion trap MS systems; ESI triple quadrupole MS systems; ESI quadrupole orthogonal TOF systems (Q-TOF); or ESI Fourier transform MS systems; etc. Peptide ion fragmentation in tandem MS (MS/MS) may be achieved using manners established in the art, such as, e.g., collision induced dissociation (CID). Algorithms and software exist in the art that compare experimental mass spectra and optionally also (partial) sequence information for the analysed peptides with a database of peptide masses/sequences predicted on the basis of sequence information in protein and nucleic acid databases, and identify the corresponding peptides: e.g., ProFound, X! Tandem, (http://prowl.rockefeller.edu), MASCOT (http://www.matrixscience.com, Matrix Science Ltd. London), Sequest (http://fields.scripps.edu/sequest/; U.S. Pat. No. 6,017,693; U.S. Pat. No. 5,538,897), OMSSA (http://pubchem.ncbi.nlm.nih.gov/omssa/), etc. Starting from the known identity of so-detected peptides, the corresponding proteins can be easily found by sequence database searching using these or other software tools. Identification of N-terminal or C-terminal peptides can also benefit from the use of specialised N-terminally or C-terminally ragged databases to account for protein processing, as known in the art (e.g., Gevaert et al. 2003. Nat Biotechnol 21: 566-569; Martens et al. 2005. Proteomics 5: 3139-3204). Generally, the herein disclosed methods may achieve identification of any number or even substantially all (i.e., comprehensive analysis) N-terminal or C-terminal peptides present in starting protein peptide mixtures. Optionally, the methods may further encompass art established technique(s) to determine the relative or absolute quantity of one or more proteins in the starting sample (see, e.g., WO 03/016861, WO 02/084250 or WO 2004/111636).

Information obtained from N-terminal or C-terminal technologies is then used for the generation of proteins with altered sensitivity towards proteases that can have modulated activity and/or longer half-lives when used for therapeutic, diagnostic or vaccination applications.

Examples of such modifications are now described in more detail in this passage:

The inventive concept is directed to the modification of the protein at its proteolytic cleavage site identified by the method of the invention, or at neighbouring positions in such a way that the proteolytic cleavage is reduced or blocked. This can either be done by chemical modification of the amino acid side chains or by introduction of specific point mutations in the nucleic acid coding sequence of the protein, at a position overlapping with or surrounding with the identified proteolytic cleavage site, leading to amino acid substitutions. In principle, any one or more of the amino acid residues at the positions surrounding the proteolytic cleavage site can be modified or substituted by any of the other 19 available amino acid residues.

Such a modification could however result in a reduction or even loss of the protein activity, which is mostly undesirable. To this end, the modification process itself can be tailored in such a way that the activity of the protein variant or modified protein is completely or at least partially preserved. In addition, such a modification should also not lead to an increase of the immunogenicity of the protein, which could result in an undesirable autoimmune response of the patient. This can for example be accomplished by introducing specific point mutations changing the amino acid sequence of the protein in question, without changing its structure in a drastic manner e.g. by substituting the amino acids with structurally related amino acids (e.g. basic, acidic, uncharged or non-polar amino acids are substituted by other respectively basic, acidic, uncharged or non-polar amino acids).

In a second approach, the amino acid side chains of the amino acid residues delineating the proteolytic cleavage site or its neighbouring amino acid residues can be modified in such a way that the protease is hampered or ineffective in the cleavage process of the protein e.g. due to sterical hindrance. These side chain modifications are less drastic than actually changing the primary structure of the protein and will more likely result in functional preservation. Oxidation, reduction, acetylation, etc. ca be used. Further examples are the modification of Arginine side chains by using HPG (p-Hydroxyphenylglyoxal), which is an Arginine selective modifying agent or of Tryptophan residues using Tryptophan side chain oxidase II.

In this respect, it is noteworthy that residues that are pointed towards the outside of the protein can be of special interest for such modification procedures, since the modification could in this case be limited to the amino acid residues extending into the outside structure of the protein, not influencing the remaining residues in the molecule.

In a further embodiment, the proteolytic cleavage of the protein of interest can also be modulated by binding of a specific binding molecule, such as an antibody, aptamer, etc, sterically hindering the binding of a protease to the proteolytic cleavage site of the protein of interest. The affinity agent or binding molecule can be binding directly to the target protein at its cleavage site, at its interaction patch with the identified protease, or to the identified protease targeting the protein of interest.

In some embodiments, one or more codon(s) of the coding sequence surrounding or overlapping with the identified cleavage site can be fully randomized i.e. all four nucleotides can occur at one or more of the positions in the codon(s). In an alternative embodiment, the randomization is limited to a subset of the four nucleotides. Such randomization can be applied to one codon only, directly situated prior to or after the identified cleavage site or surrounding the cleavage site. In an alternative embodiment, the randomization is extended to several codons overlapping with or surrounding the identified cleavage site. This randomization leads to a library of mutated nucleic acids which upon expression in a recombinant system leads to a library of mutated or modified proteins.

In a further aspect, the invention provides methods of incorporating non-natural amino acids into the synthesized proteins at or surrounding with the position of the identified proteolytic cleavage site. This technology is described for example by Sisido and coworkers (Sisido et al., 2005, FEBS Letters 579:6769-6774). This technology is further described in U.S. Pat. No. 7,045,337 by Schulz and also in U.S. Pat. No. 6,783,946 by Dix describing positively charged non-natural amino acids that closely resemble the natural amino acids lysine and arginine. The technology of Kiick described in US patent application US 2004/0058415A1 can also be of use. The advantage of this approach is that a non-natural amino acid having a similar structure as the original amino acid can be incorporated, thereby preserving the three-dimensional structure, but nonetheless inhibiting the proteolytic cleavage at said site.

The invention further provides for methods of analyzing the susceptibility of said modified protein to proteolytic cleavage and the residual activity of said modified proteins as compared to the native proteins. In a preferred embodiment, the protein variants or modified proteins of the invention are subsequently tested for their stability in vitro and in vivo in e.g. blood, and their activity in both in vitro and in vivo methods. Methods for testing protein activity are protein dependent and well known to the skilled person.

Benefits for Protein Production

It is clear that N-teromics or C-teromics technology is also able to detect protease sensitive sites during the production process. Elimination of such sites can result in benefits for production. This is especially true for proteins that are produced in the milk of e.g. transgenic animals or in other (microbial) cell-based systems wherein proteases are present. In addition, stabilized variants of such recombinant proteins that are used or that will be used for therapeutic applications can be produced, with the potential to enhance specific activity of the protein therapeutic. Reduced dosing in combination with improved production stability can significantly affect the cost of using the protein for therapy. Small molecules targeting the responsible proteases for these proteins can be valid here as well. Genetic targeting of the protease in a production host (e.g. by genetic deletion of a protease-encoding gene in E. coli) can also be envisioned as a way of improving the overall production stability.

The invention therefore provides several strategies for modifying the identified proteolytic cleavage sites thereby preventing proteolysis in vivo and creating a protein with an increased in vivo half-life and/or changed activity.

Recombinant protein production can be done by cloning the mutated coding sequence of a target protein in a vector or a plasmid and transfecting it in a biological factory, which can be an eukaryotic cell, such as e.g. a yeast cell, or which can be a prokaryotic cell such as a bacterium, depending on the complexity of the post-translational modifications that the target protein normally would undergo. Prokaryotic and eukaryotic cell systems for production of large amounts of recombinant proteins are well known in the art. One particularly preferred embodiment is the manufacturing of a recombinant protein in the milk of a transgenic animal such as a cow.

In an alternative embodiment, the mutated protein or peptide can be produced synthetically using standard peptide synthesis techniques known in the art.

The invention further encompasses a method for screening for modified or mutated proteins that 1) are functionally active or retain at least partially the activity of the native protein and 2) have a changed sensitivity towards proteolytic cleavage or processing by the protease(s) in vivo.

Proteolytic cleavage of proteins does not always result in degradation of the protein under investigation. It has to be emphasized that the use of the methods of the invention will depend on the protein envisaged. Some proteins are activated upon proteolytic cleavage, whereas other proteins are inactivated after proteolytic cleavage. The person skilled in the art would be aware of different examples for both general groups. In one embodiment, the method is therefore used to reduce the proteolytic cleavage of a protein which is inactivated by the cleavage. In an alternative embodiment, the method is used to reduce proteolytic cleavage in a protein, thereby activating the protein or increasing the protein's activity. The methods of the invention can further more be used to inactivate an inhibitor of a certain pathway, thereby (partially) activating said pathway. Alternatively, the method of the invention can be used to activate an inhibitor of a certain pathway, thereby (partially) deactivating the pathway. Similarly, the methods of the invention can be used to inactivate a protein normally activating other proteins in a certain pathway, thereby (partially) inactivating said pathway. Alternatively, the method of the invention can be used to activate a protein normally inactivating other proteins in a certain pathway, thereby (partially) activating the pathway. Non-limiting examples of such proteins are enzymes (e.g. kinases, phosphatases, lipases, oxidases, reductases, ATP/ADP-exchanging enzymes, proteases, ligases, hydrolases, transferases, lyases, isomerases, polymerases . . . ), chaperones, transcription factors, receptors ligands, transmembrane molecules, complement molecules, coagulation proteins.

In a specific embodiment, the target proteins are therapeutic proteins, meaning proteins that are used as a therapeutic agent, in which the proteolytic cleavage is modulating their activity. Changing the sensitivity of a protein towards proteolytic cleavage at said site(s) of such a protein could e.g. prolong its activity after administration to a subject, thereby benefiting its therapeutic strength. Non-limiting examples of such therapeutic proteins for which we used the method of the present invention are ApoA1 (cf. examples below), C1 inhibitor, Factor H, Antithrombin and Plasminogen (data not shown).

In another embodiment, the method of the invention can be used to normalize the activity of a hyperactive protein, by e.g. reducing the proteolytic cleavage in said protein which normally gets activated after proteolysis.

The modification of the proteolytic cleavage site of a given protein as explained in the methods of the invention, can result in complete blocking of the proteolytic cleavage or can alternatively result in a partial blocking or reduction of the proteolytic cleavage.

### Identifying Proteases and Agents Modulating Protease Activity

The invention further provides a method for identifying the protease responsible for the cleavage of wild type human proteins at their proteolytic cleavage sites identified by using the method of the invention comprising:

a) providing the isolated protein of interest,
b) providing a candidate protease,
c) analysing the proteolytic cleavage of the wild-type protein of interest at its determined proteolytic cleavage site(s) in the presence and absence of the protease using the method of the invention, thereby analysing the ability of the protease under investigation to cleave the protein of interest at its identified proteolytic cleavage position.

In addition, the invention provides a method for screening agents that inhibit the cleavage of a wild type protein of interest at its identified proteolytic cleavage position, comprising:

a) providing the isolated wild-type protein of interest
b) providing an agent
c) analyzing the proteolytic cleavage of the wild-type protein at its identified proteolytic cleavage position in the presence and absence of the agent using the method of the invention, thereby analyzing the inhibitory effect of the screened agent.

### Proteolytic Pathway Analysis

In yet a further embodiment, the invention provides a method for the analysis of proteolytic pathways comprising the detection of proteolytic cleavage in the proteins from a complex test sample, wherein the test sample is derived from a cell culture system in which a specific pathway is targeted. Examples of such pathways can be apoptosis pathways, inflammation pathways, cell-growth, -migration, -differentiation, -division or -proliferation pathways, cell signaling pathways, etc. In a typical set-up, one sub-sample of the cell culture is triggered to activate a certain pathway, while the remaining subset of cells from the sample is not. When the proteolytic cleavage profiles of both sub-samples are compared using the N-terminal or C-terminal technology of the invention, differences in proteolytic cleavage due to the induced process (e.g. apoptosis) can be identified, establishing a proteolytic scheme of events linked to a certain pathway. This data can then be used to trace mutations in subjects, which will be useful in diagnosis and prognosis of a specific disease or condition linked to the tested pathway. In one embodiment, the data regarding proteolytic cleavage in response to activation of a specific pathway can be linked to a SNP-database (cf. below) in order to detect naturally SNPs that are connected to a certain disease state and are useful in developing diagnostic tests.

### SNP Analysis

In a further embodiment, the information regarding the existence of proteolytic cleavage sites in a protein of interest can be used in combination with a single nucleotide polymorphism (SNP) database at the National Center for Biotechnology Information (NCBI) in order to investigate whether a naturally occurring mutation of the identified proteolytic cleavage site exists in the population of subjects, such as animals or humans. Single nucleotide polymorphisms (SNPs) are DNA sequence variations that occur when a single nucleotide (A,T,C, or G) in the genome sequence is changed, which occur approximately once every 100 to 300 bases. There are many techniques for SNP detection and genotyping, such as restriction fragment length polymorphism PCR(RFLP-PCR), SSCP, allele specific hybridization, primer extension, allele specific oligonucleotide ligation, sequencing.

If a search in the SNP-database, reveals a non-synonymous SNP at or surrounding the proteolytic cleavage site of the protein of interest, the invention provides for tests analysing the protease sensitivity of said naturally occurring SNP-mutant. When the mutation makes the target protein more resistant to protease activity, this information may not only be helpful for obtaining mechanistic insight in some conditions, it can also be used as a basis to develop a diagnostic test.

Further, the invention therefore provides diagnostic applications comprising the detection of a SNP-mutation in the protein of interest of a patient leading to a stabilisation or increased half-life of the protein.

In most cases, preventing proteolytic cleavage will improve the in-vivo half-life of the target protein in the subject, thereby possibly improving his condition. In one such embodiment, the mutation is detected on the protein level. In a further embodiment, the mutation is detected on the nucleotide level. The carriers of this allele can be easily identified by genetic screening using e.g. PCR analysis of their genetic material obtained from a blood sample.

### Gene Therapy

In gene therapy, either a normal or a modified gene can be inserted into the genome to replace an existing gene, being either an abnormal or a disease-causing gene or a normal gene which is e.g. prone to proteolytic cleavage by a protease. In the first example, the disease-causing gene can be a gene which has an additional proteolytic cleavage site, which was identified in a subject having a specific disease by the method of the invention. This disease-causing gene is then replaced by a gene, wherein this additional proteolytic cleavage site is altered in order to change its sensitivity towards proteolysis. In the second example, a normal gene, which is normally prone to proteolytic cleavage can be altered in such a way that it is no longer prone to proteolytic cleavage by a point-mutation in or surrounding the proteolytic cleavage site identified by the method of the invention, thereby possibly altering the half-life or activity of said protein.

A carrier or vector is used to deliver the therapeutic gene to the subject's target cells. The most common type of vectors are viruses that have been genetically altered to carry a specific DNA or RNA molecule. Target cells are infected with the vector, which then unloads its genetic material containing the specific DNA or RNA molecule into the target cell. The generation of a functional protein product from the RNA or DNA can e.g. restore the functioning of the target cell to a normal state. The specific DNA or RNA molecule may be inserted into a nonspecific location within the genome to replace a nonfunctional gene. This approach is most common. In addition, a gene could be swapped for a modified gene through homologous recombination. Alternatively, the gene could be repaired through selective reverse mutation, which returns the gene to its normal function. The regulation (the degree to which a gene is turned on or off) of a particular gene could also be altered.

Some examples of carrier viruses are retroviruses, adenoviruses, Adeno-associated viruses, from the parvovirus family and Lentiviruses.

In a specific embodiment, the method of the invention uses the so called integrase-defective lentiviral vectors (IDLY), for sequence specific gene editing (cf. Lombardo et al., Nat. Biotechnol. 2007 November; 25(11):1298-306).

Non-viral methods can also be used such as administering of naked DNA in the form of plasmids or naked PCR products. More efficient methods for delivery of the naked DNA are electroporation and the use of a so called gene gun, which shoots DNA coated gold particles into the cell using high pressure gas. The use of synthetic oligonucleotides in gene therapy is to inactivate the genes involved in the disease process. There are several methods by which this is achieved. One strategy uses antisense specific to the target gene to disrupt the transcription of the abnormal gene. Another uses small molecules of RNA called siRNA to signal the cell to cleave specific unique sequences in the mRNA transcript of the abnormal gene, disrupting translation of the abnormal mRNA, and therefore expression of the gene. A further strategy uses double stranded oligodeoxynucleotides as a decoy for the transcription factors that are required to activate the transcription of the target gene. The transcription factors bind to the decoys instead of the promoter of the abnormal gene, which reduces the transcription of the target gene, lowering expression. To improve the delivery of the new DNA into the cell, the DNA must be protected from damage and its entry into the cell must be facilitated. To this end, molecules such as lipoplexes, polyplexes and dendrimers can be used that have the ability to protect the DNA from undesirable degradation during the transfection process.

Alternatively, the gene therapy can be effected through the ex vivo transfection of target cells isolated form a patient (e.g. white blood cells, endothelial cells, islet cells, stem cells, etc.) with the plasmid of choice, which are then transplanted back into the subject after modification and can start producing the (modified) protein of choice in the subject. The term “target cells” implies all possible cell types that express the protein of interest. The therapy can be effected in a non-cell-specific way or can be directed to a specific target cell-type, depending on the disease or condition of the subject.

One specific example of gene therapy in combination with the method of the present invention is the selective site-directed mutagenesis of a sequence encoding a protein of interest, wherein the proteolytic cleavage site identified by the method is mutated.

The methods of the present invention can also be used to compare the proteolytic cleavage events in diseased versus healthy patients and the results thereof can be used to change or repair a certain gene in such a way that the abnormal proteolytic events are restored to a normal level. In one such example, a disease or condition can be caused by the unwanted cleavage of a certain protein, thereby reducing its functionality. Altering the proteolytic cleavage site in the disease-causing protein could restore its function and benefit the patient. Alternatively, a protein can be given a prolonged activity or half-life by reducing its proteolytic cleavage, benefiting a patient in need of such an altered protein activity.

### DEFINITIONS

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. When specific terms are defined in connection with a particular aspect or embodiment, such connotation is meant to apply throughout this specification, i.e., also in the context of other aspects or embodiments, unless otherwise defined.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

All documents cited in the present specification are hereby incorporated by reference in their entirety.

The term “protein” as used herein refers to naturally or recombinantly produced macromolecules comprising one or more polypeptide chains, i.e., polymeric chains of amino acid residues linked by peptide bonds. The term thus encompasses monomeric proteins, as well as protein dimers (hetero-as well as homo-dimers) and protein multimers (hetero-as well as homo-multimers). Further, the term also encompasses proteins that carry one or more co- or post-expression modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. In addition, the term includes nascent protein chains as well as partly or wholly folded proteins, misfolded proteins, partly or wholly unfolded or denatured proteins, and may also cover coalesced or aggregated proteins, in particular where the latter are amenable to proteolysis. The term further also includes protein variants or mutants which carry amino acid sequence variations vis-à-vis a corresponding native protein, such as, e.g., amino acid deletions, additions and/or substitutions. The term contemplates both full-length proteins and protein parts, preferably naturally-occurring protein parts that ensue from further processing of said full-length proteins.

The invention may analyse a single protein (e.g., gel-excised protein) and is particularly suitable for analysing mixtures of proteins, including complex protein mixtures. The terms “mixture of proteins” or “protein mixture” generally refer to a mixture of two or more different proteins, e.g., a composition comprising said two or more different proteins.

In preferred embodiments, a mixture of proteins to be analysed herein may include more than about 10, preferably more than about 50, even more preferably more than about 100, yet more preferably more than about 500 different proteins, such as, e.g., more than about 1000 or more than about 5000 different proteins.

An exemplary complex protein mixture may involve, without limitation, all or a fraction of proteins present in a biological sample or part thereof. The terms “biological sample” or “sample” as used herein generally refer to material, in a non-purified or purified form, obtained from a biological source. By means of example and not limitation, samples may be obtained from: viruses, e.g., viruses of prokaryotic or eukaryotic hosts; prokaryotic cells, e.g., bacteria or archeae, e.g., free-living or planktonic prokaryotes or colonies or bio-films comprising prokaryotes; eukaryotic cells or organelles thereof, including eukaryotic cells obtained from in vivo or in situ or cultured in vitro; eukaryotic tissues or organisms, e.g., cell-containing or cell-free samples from eukaryotic tissues or organisms; eukaryotes may comprise protists, e.g., protozoa or algae, fungi, e.g., yeasts or molds, plants and animals, e.g., mammals, humans or non-human mammals. Biological sample may thus encompass, for instance, a cell, tissue, organism, or extracts thereof. A biological sample may be preferably removed from its biological source, e.g., from an animal such as mammal, human or non-human mammal, by suitable methods, such as, without limitation, collection or drawing of body fluid samples such as blood, plasma, serum, urine, saliva, cerebrospinal fluid, nipple aspirate, milk, ductal lavage, sweat or perspiration, tumor exudates, joint fluid (e.g. synovial fluid), inflammation fluid, tears, semen and vaginal secretions, sputum, mucus, faeces, etc., drawing of blood, cerebrospinal fluid, interstitial fluid, optic fluid (vitreous) or synovial fluid, or by tissue biopsy, resection, etc. A biological sample may be further subdivided to isolate or enrich for parts thereof to be used for obtaining proteins for analysing in the invention. By means of example and not limitation, diverse tissue types may be separated from each other; specific cell types or cell phenotypes may be isolated from a sample, e.g., using FACS sorting, antibody panning, laser-capture dissection, etc.; cells may be separated from interstitial fluid, e.g., blood cells may be separated from blood plasma or serum; or the like. The sample can be applied to the methods of the invention directly or can be processed, extracted or purified to varying degrees before being used. The sample can be derived from a healthy subject or a subject suffering from a condition, disorder, disease or infection. For example, without limitation, the subject may be a healthy animal, e.g., human or non-human mammal, or an animal, e.g., human or non-human mammal, that has cancer, an inflammatory disease, autoimmune disease, metabolic disease, CNS disease, ocular disease, cardiac disease, pulmonary disease, hepatic disease, gastrointestinal disease, neurodegenerative disease, genetic disease, infectious disease or viral infection, or other ailment(s).

Preferably, protein mixtures derived from biological samples may be treated to deplete highly abundant proteins there from, in order to increase the sensitivity and performance of proteome analyses. By means of example, mammalian samples such as human serum or plasma samples may include abundant proteins, inter alia albumin, IgG, antitrypsin, IgA, transferrin, haptoglobin and fibrinogen, which may preferably be so-depleted from the samples. Methods and systems for removal of abundant proteins are known, such as, e.g., immuno-affinity depletion, and frequently commercially available, e.g., Multiple Affinity Removal System (MARS-7, MARS-14) from Agilent Technologies (Santa Clara, Calif.).

The term “protein peptide mixture” generally refers to a mixture of peptides derived from a protein or preferably from a mixture of two or more different proteins (i.e., protein mixture).

The terms “peptide” or “protein peptide” as used herein generally refer to fragments of a protein derived by fragmentation of said protein or of any one or more of its polypeptide chains, into two or more fragments. While the terms encompass peptides of all sizes and molecular weights, peptides and protein peptide mixtures preferred in the invention may have average and/or median length of less than about 100 amino acids, e.g., less than about 90 amino acids, less than about 80 amino acids, more preferably less than about 70 amino acids or less than about 60 amino acids, even more preferably less than about 50 amino acids, e.g., particularly preferably less than about 40 amino acids or less than about 30 amino acids. In further embodiments, peptides and protein peptide mixtures preferred in the invention may have average and/or median length of at least about 5 amino acids, preferably at least about 10 amino acids, even more preferably at least about 15 amino acids, e.g., at least about 20 amino acids. Hence, in yet further embodiments, peptides and protein peptide mixtures preferred in the invention may have average and/or median length of between about 5 and about 100 amino acids, preferably between about 5 and about 50 amino acids e.g., between about 5 and about 40 amino acids or between about 5 and about 30 amino acids, more preferably between about 5 and about 20 amino acids Such peptide sizes may be particularly amenable to proteome analysis.

As used herein, the term “functional assay” refers to an assay that provides an indication or a measurement of the activity of a protein. In certain embodiment, where the protein is an enzyme, the assay involves determining the effectiveness of the variant protein in catalyzing the reaction the native protein is known to catalyze. In another embodiment, the activity of the protein can be phosphorylation or dephosphorylation, inhibition or activation of another protein, binding to another protein or a receptor, thereby inducing a signal, the release or storage of energy, fat, sugars, or other compounds, the inhibition or stimulation of DNA-replication, the inhibition or stimulation of apoptosis, necrosis, cell-growth, cell-division, cell-migration, cell-signaling, or any other known function.

The term “cleavage assay” implies any assay that is indicative of the amount of proteolytic cleavage or processing of a target protein. In a preferred embodiment of the invention, the assay is used on both a native protein preparation and a modified protein or protein variant, in order to establish improved stability towards proteolytic cleavage of the protein variant or modified protein of the invention. In some cases, the specific protease responsible for the proteolysis of a target protein may be unknown. In such a case, proteolysis in the assay can be simulated by using a fresh sample from a subject, for which it is clear that proteolysis occurs.

As used herein, the term “binding molecule” or “affinity agent” or “tagging agent” or “labelling agent” refers amongst others to antibodies such as, single-chain antibodies (nanobodies), monoclonal antibodies, polyclonal antibodies and antibody fragments thereof, aptamers, photoaptamers, specific interacting proteins, ligands, Molecular Imprinting Polymers (MIPs), Designed Ankyrin Repeat Proteins (DARPins) and the like. Antibodies and their fragments can be isolated or manufactured according to known protocols.

Aptamers that bind specifically to the biomarkers of the invention can be obtained using the so called SELEX or Systematic Evolution of Ligands by EXponential enrichment. In this system, multiple rounds of selection and amplification can be used to select for DNA or RNA molecules with high specificity for a target of choice, developed by Larry Gold and coworkers and described in U.S. Pat. No. 6,329,145. Recently a more refined method of designing co-called photoaptamers with even higher specificity has been described in U.S. Pat. No. 6,458,539 by the group of Larry Gold.

Methods of identifying binding agents such as interacting proteins and small molecules are also known in the art. Examples are two-hybrid analysis, phage display, ribosome display, immunoprecipitation methods and the like.

Molecularly imprinted polymers (MIPs) are synthetic polymers having a predetermined selectivity for a given analyte, or group of structurally related compounds, that make them ideal materials to be used in separation processes such as molecularly imprinted solid-phase extraction (MISPE) or immuno-type assays (MIA). Examples of MIPs include aptamers and bio-nanocomposites. MIPs are prepared by allowing a network polymer to form in presence of a template, i.e. a structure directing agent. Removal of the template postpolymerization leaves behind a cavity which is complementary to the template in terms of size and shape. Thus MIPs can be programmed to recognize a large variety of low molecular target structures often associated with antibody-like affinities and selectivities.

DARPins can be developed according to well known techniques, as described by Stumpp and Amstutz (Curr Opin Drug Discov Devel. 2007 March; 10(2):153-9). DARPins are based on natural repeat proteins and are very stable and well-expressed in bacteria and, since they do not contain any cysteines, they remain fully active in the cellular cytoplasm.

For reasons of clarity, the term “true N- or C-terminus” means the actual starting point of the mature protein, e.g. after processing or cleavage of the pro or pre-pro domains. On the other hand, the terms “genuine N- or C-termini”, “novel internal N- or C-termini” and “naturally occurring N- or C-termini” can be used interchangeably throughout the specification. In all cases, the terms mean all N- or C-termini, formed after proteolysis.

As used herein, the term “target protein” refers to any possible target protein for which stabilization could be desired. Examples of target proteins may be: enzymes, receptors, ligands, extracellular proteins, biomarkers useful in diagnosis, reporter proteins, proteins useful in vaccines or other pharmaceuticals and the like.

The terms “condition”, “disease” or “phenotype” used throughout the invention may be a pathological condition of interest in patients, such as, e.g., cancer, an inflammatory disease, autoimmune disease, metabolic disease, CNS disease, ocular disease, cardiac disease, pulmonary disease, hepatic disease, gastrointestinal disease, neurodegenerative disease, genetic disease, infectious disease or viral infection; vis-à-vis the absence of such condition in healthy controls. Other comparisons may be envisaged between samples from, e.g., stressed vs. non-stressed conditions/subjects, drug-treated vs. non drug-treated conditions/subjects, benign vs. malignant diseases, adherent vs. non-adherent conditions, infected vs. uninfected conditions/subjects, transformed vs. untransformed cells or tissues, different stages of development, conditions of overexpression vs. normal expression of one or more genes, conditions of silencing or knock-out vs. normal expression of one or more genes, and so on.

### N-Terminal and C-Terminal Technology

The invention provides methods for isolating N-terminal peptides from a protein or mixture of proteins, comprising:

(a) protecting the N-terminal amino acid in the protein or in proteins of the protein mixture,
(b) fragmenting the protein or the protein mixture from (a) to obtain a protein peptide mixture, and
(c) reacting the protein peptide mixture from (b) with an aminopeptidase, whereby said N-terminal peptides are isolated.

This aspect takes advantage of the situation that fragmentation of a protein in which the N-terminal amino acid has been suitably protected prior to said fragmentation will generate an N-terminal peptide containing a protected N-terminal amino acid, and a C-terminal peptide and optionally one or more internal peptides containing an unprotected amino acid at their respective, newly generated N-termini. Consequently, reacting the protein peptide mixture obtained by fragmentation of said protein with an aminopeptidase leads to hydrolysis (degradation) of the unprotected C-terminal and internal peptides progressively from their respective N-termini into their constituent amino acids. The protected N-terminal peptides of the protein are not degraded by said aminopeptidase, and thereby become enriched or isolated and can be used for downstream analysis.

In another aspect the invention provides a method for isolating C-terminal peptides from a protein or mixture of proteins, comprising:

(a) protecting the C-terminal amino acid in the protein or in proteins of the protein mixture,
(b) fragmenting the protein or the protein mixture from (a) to obtain a protein peptide mixture, and
(c) reacting the protein peptide mixture from (b) with a carboxypeptidase, whereby said C-terminal peptides are isolated.

This aspect takes advantage of the situation that fragmentation of a protein in which the C-terminal amino acid has been suitably protected prior to said fragmentation will generate a C-terminal peptide containing a protected C-terminal amino acid, and an N-terminal peptide and optionally one or more internal peptides containing an unprotected amino acid at their respective, newly generated C-termini. Hence, reacting the protein peptide mixture obtained by fragmentation of said protein with a carboxypeptidase leads to hydrolysis (degradation) of the unprotected N-terminal and internal peptides progressively from their respective C-termini into their constituent amino acids. The protected C-terminal peptides of the protein are not degraded by said carboxypeptidase, and thereby become enriched or isolated and can be used for downstream analysis.

Advantageously, the highly efficient and processive action of aminopeptidases and carboxypeptidases can ensure robust, reliable and substantially complete removal of the unprotected peptides, and thereby achieve high degree of enrichment or isolation of, respectively, the protected N-terminal or C-terminal peptides of the starting proteins. Moreover, enzymatic hydrolysis used in the present invention is fairly easy to perform and avoids the need for chemical modifications, which may be rather susceptible to reaction conditions. Also, whereas previous methods relying on separation of peptides labelled with a given moiety from peptides not so-labelled were dependent on the specificity of means of said separation, the present methods degrade the unwanted peptides to their constituent amino acids which substantially do not interfere with downstream analysis of the desired N-terminal or C-terminal peptides.

Moreover, if required the amino acids resulting from hydrolysis of the unwanted peptides may be readily separated and removed from the desired N-terminal or C-terminal peptides by common techniques, such as for example RP-chromatography or size exclusion chromatography, on the basis of their different properties, such as, e.g., their considerably smaller size or molecular weight in comparison with peptides).

Hence, the present peptide isolation methods provide robust and straightforward means to isolate or enrich N-terminal or C-terminal peptides from protein peptide mixtures, such as from complex protein peptide mixtures representative of biological samples.

In related aspects, the present methods may be tailored to isolate N-terminal peptides or C-terminal peptides from proteins which are suitably altered in vivo, in case their newly formed N-termini after internal proteolytic cleavage are in vivo modified. For example, a considerable portion or even the majority of cellular proteins in mammalian cells may be in vivo acetylated on their N-terminal α-NH₂ group. In another example, proteins of prokaryotes are translated with a formylated methionine as an initiator for translation, and although the formyl group is typically removed during the translation, it can still be found in some proteins. The deformylase enzyme catalysing the formyl group removal is targeted by next generation antibiotics, underlying the value of tools for monitoring protein deformylation. In yet another example, the activity of glutaminyl cyclase (EC 2.3.2.5) results in the formation of pyroglutaminyl peptides, which is cyclised form of the N-terminal glutamine on some peptides. Activity of this enzyme is described in organ tissues like brain, pituitary, adrenal gland and lymphocytes (Busby et al., 1987, J Biol Chem, 262/15, 8532). These pyroglutaminyl peptides do not have a free amino-terminal amine and they may thus be protected form aminopeptidase activity. In a further example, protein introns or inteins derived from protein splicing include a cyclisation of asparagine (Asn) on their C-terminus. Also in an example, cholesterol modification can occur at the C-terminus and can be sometimes transferred to the C-terminus of an intein of a hedgehog protein.

Accordingly, in an aspect the invention provides a method for isolating, from a protein or mixture of proteins, N-terminal peptides in which the N-terminal amino acid has been blocked in vivo, comprising: (i) fragmenting the protein or the protein mixture to obtain a protein peptide mixture, and (ii) reacting the protein peptide mixture from (i) with an aminopeptidase, whereby said N-terminal peptides in which the N-terminal amino acid has been blocked in vivo are isolated.

In another aspect the invention provides a method for isolating, from a protein or mixture of proteins, C-terminal peptides in which the C-terminal amino acid has been blocked in vivo, comprising: (i) fragmenting the protein or the protein mixture to obtain a protein peptide mixture, and (ii) reacting the protein peptide mixture from (i) with a carboxypeptidase, whereby said C-terminal peptides in which the C-terminal amino acid has been blocked in vivo are isolated.

The term “blocked in vivo” denotes any in vivo modification of a protein's N-terminal or C-terminal amino acid, which can prevent the cleaving-off of said N-terminal or C-terminal amino acid from a protein or peptide containing it by the action of aminopeptidase or carboxypeptidase, respectively. The term “in vivo” generally refers to a living biological system such as, e.g., a cell, a tissue, an organ or an organism, whether in native surroundings or isolated there from (e.g., cell culture). Particularly preferred, although non-limiting, types of in vivo alterations include N-terminal α-NH₂ acetylation or N-terminal formylation of proteins, which can prevent the action of aminopeptidase on the respective N-terminal peptides; or C-terminal Asn cyclisation or C-terminal cholesterol addition of proteins which can prevent the action of carboxypeptidase on their respective C-terminal peptides.

As already noted, the present methods can enrich N-terminal or C-terminal peptides that correspond to the N-termini or C-termini of respective full-length proteins, and can also recover N-terminal or C-terminal peptides which correspond to—and thereby identify—proteolytic cleavage events within (full-length) proteins. For example, protein processing or degradation in vivo may produce protein fragments displaying novel N-terminal ends and/or C-terminal ends. The above methods can advantageously follow the appearance of such novel N-terminal or C-terminal peptides which can be identified and may be indicative of novel proteolytic processing events, and/or can follow the changes in absolute or relative quantity of known N-terminal or C-terminal peptides, representative of known cleavage events. Accordingly, the present methods may be advantageously employed in the proteomic study of protein processing (“degradomics”).

By means of example and not limitation, a general approach to identify N-terminal or C-terminal peptides corresponding to proteolytic processing sites may encompass isolating N-terminal or C-terminal peptides of proteins as taught herein, and identifying among so-isolated peptides those which correspond to internal portions of known or predicted full-length proteins.

In further aspects, the subset of N-terminal peptides or C-terminal peptides isolated as taught here above can be subjected to downstream proteome analyses to identify one or more constituent peptides and their corresponding proteins. Typically, this may entail acquiring relevant information for the isolated N-terminal peptides or C-terminal peptides—principally peptide mass and preferably also (partial) peptide sequence—which information allows for database searching to identify the peptides and trace them back to their parent proteins. Accordingly, in an aspect, the methods of the invention may further comprise identifying one or more of the isolated N-terminal peptides or C-terminal peptides, whereby said identified N-terminal peptides or C-terminal peptides represent one or more proteins from the mixture of proteins.

However, given that the complexity of the isolated N-terminal peptides or C-terminal peptides may still be considerable, said peptide identification step may preferably be preceded by a further separation (fractionation) of the peptides using a single- or multi-dimensional separation process. This can further improve the reliability of peptide identification. Accordingly, in an aspect, the methods of the invention may further comprise: (i) separating the isolated N-terminal peptides or C-terminal peptides into fractions of peptides via a single- or multi-dimensional separation process; and (ii) identifying one or more N-terminal peptides or C-terminal peptides from one or more of said fractions, whereby said identified N-terminal peptides or C-terminal peptides represent one or more proteins from the mixture of proteins.

The separation process may resolve the peptides on the basis of one or more physical and/or chemical properties. Exemplary physical and/or chemical properties based on which peptides can be resolved include, without limitation, net charge, electrophoretic mobility (EPM), isoelectric point (pI), molecular size and/or ability or tendency to form certain type(s) of molecular interactions, such as, e.g., hydrogen bonding, dispersive interactions, dipole-dipole polar interactions, dipole-induced dipole polar interactions, ionic interactions, hydrophobic interactions, etc.

Such properties may be evaluated using a variety of separation techniques known per se, including inter alia various electrophoretic and chromatographic separation methods. Preferably, the separation process may comprise or consist of chromatography, such as 1D-, 2D-, 3D- or higher-dimensional chromatography, preferably 1D- or 2D-chromatography, more preferably liquid chromatography.

It shall be appreciated that in the present methods the protein peptide mixture may be treated with aminopeptidase or carboxypeptidase, thereby enriching for N-terminal or C-terminal peptides, respectively, and only thereafter subjected to the above described separation (fractionation) step. This simplifies the handling, since the digest with the aminopeptidase or carboxypeptidase can be performed in a single reaction on the whole protein peptide mixture.

Accordingly, in an aspect the invention provides a method for N-terminal peptide and protein identification and optionally quantification from a mixture of proteins comprising: (a) protecting the N-terminal amino acid in proteins of the protein mixture; (b) fragmenting the protein mixture from (a) to obtain a protein peptide mixture; (c) reacting the protein peptide mixture from (b) with an aminopeptidase, thereby isolating N-terminal peptides; (d) separating the isolated N-terminal peptides into fractions of peptides via a single- or multi-dimensional separation process; and (e) identifying and optionally quantifying one or more N-terminal peptides from one or more of said fractions, whereby said identified N-terminal peptides represent one or more proteins from the mixture of proteins.

Also, in an aspect the invention provides a method for C-terminal peptide and protein identification and optionally quantification from a mixture of proteins comprising: (a) protecting the C-terminal amino acid in proteins of the protein mixture; (b) fragmenting the protein mixture from (a) to obtain a protein peptide mixture; (c) reacting the protein peptide mixture from (b) with a carboxypeptidase, thereby isolating C-terminal peptides; (d) separating the isolated C-terminal peptides into fractions of peptides via a single- or multi-dimensional separation process; and (e) identifying and optionally quantifying one or more C-terminal peptides from one or more of said fractions, whereby said identified C-terminal peptides represent one or more proteins from the mixture of proteins.

Otherwise, it is also contemplated to first separate (fractionate) the protein peptide mixture into fractions of peptides using the above described separation step, and only thereafter treat said fraction(s) with aminopeptidase or carboxypeptidase to isolate N-terminal peptides or C-terminal peptides there from, respectively. Such sequence of actions may, e.g., allow to perform the reaction with amino- or carboxypeptidase on a limited number of fractions of interest, thereby reducing the reaction volumes and need for reagents.

Accordingly, in an aspect the invention provides a method for N-terminal peptide and protein identification and optionally quantification from a mixture of proteins comprising: (x) protecting the N-terminal amino acid in proteins of the protein mixture; (y) fragmenting the protein mixture from (x) to obtain a protein peptide mixture; (z) separating the protein peptide mixture from (y) into fractions of peptides via a single- or multi-dimensional separation process; (u) reacting one or more fractions from (z) with an aminopeptidase, thereby isolating N-terminal peptides; and (w) identifying and optionally quantifying one or more N-terminal peptides from one or more fractions of (u), whereby said identified N-terminal peptides represent one or more proteins from the mixture of proteins.

Also, in an aspect the invention provides a method for C-terminal peptide and protein identification and optionally quantification from a mixture of proteins comprising: (x) protecting the C-terminal amino acid in proteins of the protein mixture; (y) fragmenting the protein mixture from (x) to obtain a protein peptide mixture; (z) separating the protein peptide mixture from (y) into fractions of peptides via a single- or multi-dimensional separation process; (u) reacting one or more fractions from (z) with a carboxypeptidase, thereby isolating C-terminal peptides; and (w) identifying and optionally quantifying one or more C-terminal peptides from one or more fractions of (u), whereby said identified C-terminal peptides represent one or more proteins from the mixture of proteins.

The invention also provides a kit for identifying a proteolytic cleavage site in a protein comprising;

a) an N-terminal capping agent
b) a proteolytic enzyme
c) an antibody, aptamers or other binding molecule specific for the protein under investigation such as Cl inhibitor, Fibrinogen, Factor H, Antithrombin, Plasminogen and ApoA1.

The term “surrounding” used herein should be interpreted as being either one of 0, 1, 2, or 3 amino acid positions before or after the indicated amino acid position.

### EXAMPLES

### Example 1

### N-Terminal Analysis to Detect ApoA1 Processing

The COFRADIC N-terminal proteomics platform allows us to specifically analyse the N-terminus of a protein (or of a number of proteins), but in addition application of such a strategy also reveals proteolytic processing as these novel N-termini are also readily detected. Application of this platform on a serum sample revealed the occurrence of a novel cleavage event in human Apolipoprotein A1 (Swissprot accession: APOA1_HUMAN) after position R184.

### Protocol:

### Delipidation and Affinity Removal of 6 Abundant Proteins

Serum from a healthy volunteer was prepared according to standard procedures. One volume of TBS (100 mM Tris-HCl ph7.4, 150 mM NaCl) and one volume of trichlorotrifluoroethane (Riedel-de-Haen, #34874) are added to a 120 μl serum sample. After vortexing and centrifugation, the delipidated sample is transferred to a new vial and diluted 2.5× with MARS buffer A complemented with protease inhibitors. The sample is depleted with MARS level I column according to the manufacturer's description (Multiple Affinity Removal System LevelI, Agilent).

### Reduction/Alkylation and Acetylation

Guanidiniumhydrochloride (GdnHCl) is added to the depleted sample to obtain a final concentration of 3M. TCEP (TCEP.HCl, Pierce #20490) is added at 25 molar excess (when average size of proteins is expected to be 30 kDa) to reduce S—S bridges and the sample was incubated for 10′ at 30° C. Iodoacetamide (Fluka #57670) for alkylation of sulfhydryl groups was added at a 50 molar excess and left for 60′ at 30° C. Desalting was performed using PD10 columns resulting in a buffer swap to 50 mM sodiumphosphate pH 8.0, 1.4 M GdnHCl. For blocking of N-termini sulfo-NHS-acetate (Pierce #26777) is added to the sample at a 75 molar excess for 90′ at 30° C. NH₂OH is used to remove unwanted side reactions with serine, threonine and tyrosine (3 molar excess compared to sulfo-NHS-acetate). The sample was desalted again by PD10 to 10 mM NH₄HCO₃ buffer. Trypsin digestion was performed after heating the sample to 99° C. for 5′. A 50:1 (w:w) substrate:trypsin ratio was employed for ON digestion at 37° C.

### COFRADIC Primary and Secondary Runs, Nano-LC Fractionations

An estimated 500 μg of peptide material in 3M GdnHCl was acidified with TFA and was fractionated in the primary run to 12 fractions using C18 Reverse Phase columns (Zorbax 300SB-C18, Agilent) on a HPLC1100 series instrument. All fractions were subsequently dried by vacuum centrifugation at 50° C., re-dissolved in 50 μl 100 mM Borate solution pH 9.5 and treated 3 times with tri-nitro-benzene-sulfonic acid (10 μl of 15 mM TNBS stock, Fluka #92822) to modify internal non-blocked peptides. The 12 collected fractions were run again under identical conditions to separate the TNP-peptides from the unblocked peptides. 32 fractions were collected for each primary run, resulting in a total of 384 fractions for this secondary run. These 384 peptide fractions were pooled in 48 fractions after adding solvent

A (0.1% FA) according to a scheme for maximal separation. These fractions were then used for NanoLC separation using an Ultimate 3000 system (Dionex) equipped with a C18 PepMap 100 column. Direct spotting on MALDI targets was realized with a Probot system (Dionex). CHCA matrix α-cyano-4-hydroxy-cinnamic acid, Laser Bio Lab # M101) and internal standard peptides (Proteomix, Laser Bio Lab # C104) were added to the flow for optimal matrix crystal formation. Maldi targets were provided by Applied Biosystems (Opti-TOF LC/MALDI insert, # 1018469). 198 spots for each of the 48 fractions resulted in a total of 12 MALDI target plates.

### MS/MS Analysis and Search Settings

MS and MS/MS measurements were performed on a 4800 MALDI-TOF/TOF machine (Applied Biosystems) in the positive reflectron mode using internal calibration. The scan range for the MS spectra stretched from 500-4000Da. A list of the top 20 signals, per MS spectrum was generated and MS/MS experiments were performed under “metastable precursor on” conditions, without the use of CID (collision induced dissociation) and at 1 keV. The precursor mass window was set at a resolution of 250 FWHM (full width half maximum). Unfiltered MASCOT generic files (mgf) were subsequently searched against both standard and ragged human Sprot databases using MASCOT as search engine. The latter database was used to detect N-terminally ragged peptides which are abundantly present in serum. As search settings for MASCOT, we used as variable modifications pyro forms of glutamine, asparagine and cysteine, methionine oxidation and acetylation at the N-terminus, and as fixed modifications alkylated cysteine and acetylated lysine. Only peptides ranking #1 with scores above the 95% probability threshold were withheld. Spectra that had multiple peptide hits above the probability threshold were regarded as unidentified. Random hits were determined by searching the data against randomized databases. Proteins were reported if they had at least 1 peptide that unequivocally defines it.

### Result:

The numbers of identified acetylated peptides within ApoA1 were mapped on the ApoA1 sequence as it was entered in the SwissProt database (ApoA1_HUMAN). The number of identifications in the described platform can be considered a semi-quantitative readout for abundance of the different (proteolytic) variants within a protein. However this number also reflects retention (spreading of the peptide on the columns) and ionisation/fragmentation behaviour of the identified peptides, and is thus not an ideal measure.

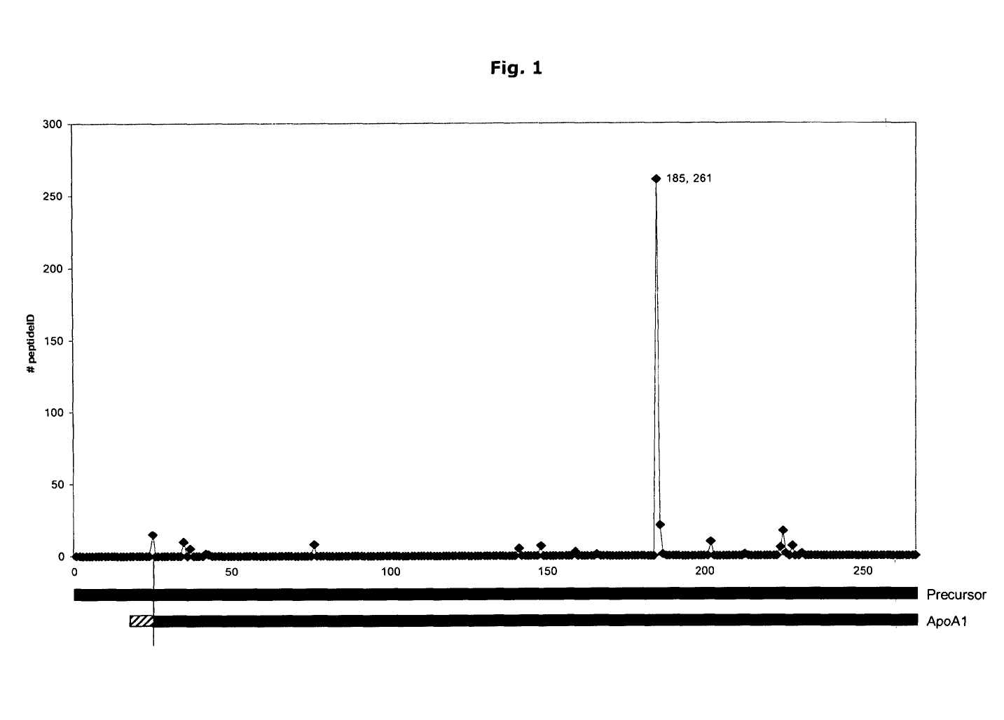

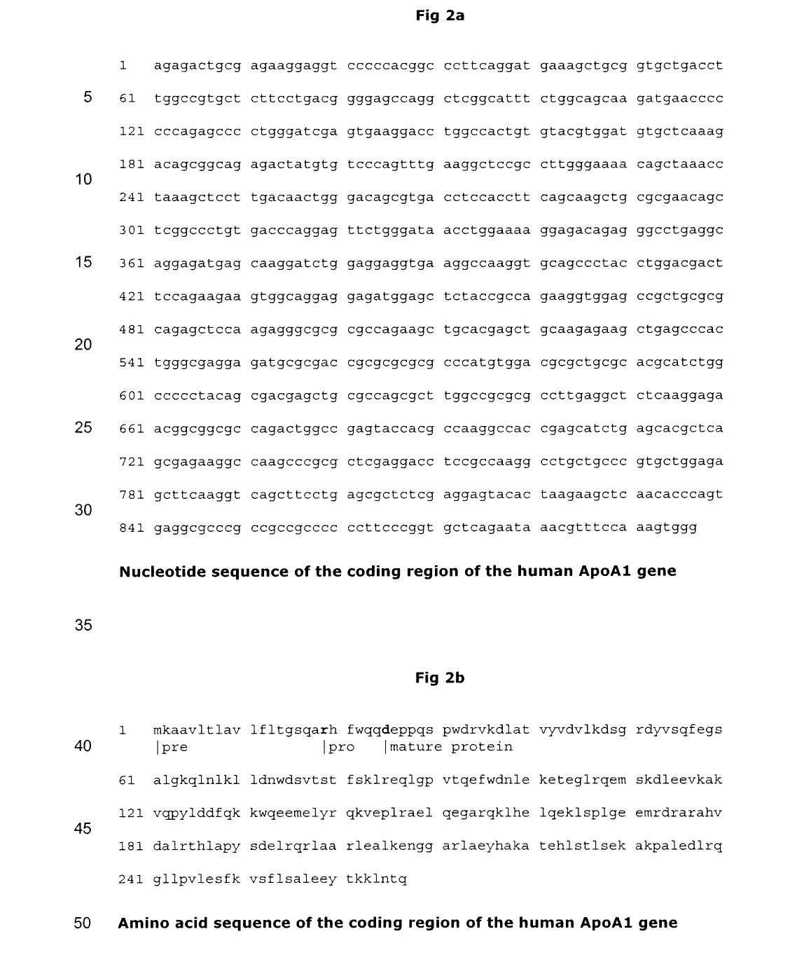

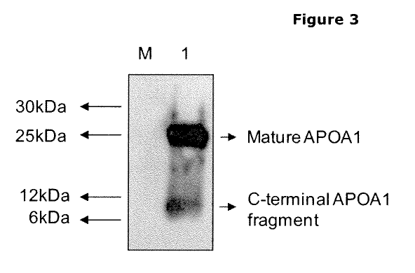

We identify the N-terminus of the processed ApoA1 protein starting at position 25 (after removal of the signal and the propeptide). In addition, we have also identified an acetylated peptide starting at position 185 for a high number of times, suggesting extensive cleavage of the protein at this site (FIG. 1).

### Example 2a

### Western Analysis for ApoA1 Variants in Blood of a Healthy Person

Using antibodies directed against the N- and C-terminus of ApoA1 in Western analysis, we are able to identify the naturally occurring variants of this protein that can be found in the blood of a normal healthy person. This confirms the data obtained with the N-terminal discovery platform described higher.

### Protocol:

Serum samples from healthy donors (appr. 2 μl or 100 mg) are diluted to 16 ul with Phosphate Buffered Saline (0.2 M Phosphate pH7.4, 150 mM NaCl). 4 μl 5× Loading buffer is added (0.313 M Tris-HCl pH 6.8, 10% SDS, 0.05% bromophenol blue, 50% glycerol). The sample is heated to 99° C., and after cooling loaded on 15% SDS-PAGE gels (Biorad, Tris-HCl ready gel). After separation, the protein material is transferred to Immobilon-P^{SQ} (Millipore) membranes by electroblotting. Membranes are subsequently blocked by milk powder (2%) dissolved in TBS-T (100 mM Tris-HCl pH7.4; 150 mM NaCl; 1/1000 Tween20) for 30 min. to 1 hr. The EP1368 antibody is diluted 1/20000 in TBS-T milk, and used for incubation of the protein blot for 1 hr at RT. The ab33470 is diluted to 1.5 μg/ml in TBS-T milk and left for 1 hr at RT on the blot. After washing with TBS-T (4×5 min.), secondary antibody (Donkey polyclonal to rabbit IgG, HRP coupled, ab16284) is diluted 1/2500 in TBS-T milk, and left on the blot for 30 min. to 1 hr. After washing with TBS-T (4×5 min.), the blot is developed using Amersham Hyperfilm™ ECL Bioscreen, Hypercassette™ (both from Amersham Biosciences) and standard photo development equipment (Kodak AL4).

### Results:

At least 2 species are detected: the full length form of about 30 kDa and a processed C-terminal fragment of about 10 kDa. This observation implies that the processing of ApoAI does not occur post-sampling, but rather in vivo. In plasma, the N-terminus starting at position 185 can also be detected (see higher), suggesting that processing does not occur during serum preparation.

### Example 2b

### Immunological Detection of the C-Terminal Fragment in ApoA-1

As an alternative detection system to show processing of ApoA-1, we used a classic immunological approach. The AI-4.1 mouse monoclonal antibody detects specifically the ApoA-1 sequence and was obtained by immunization of BALB/c mice with the C-terminal fragment of ApoA-1 obtained after cyanogen bromide cleavage of the protein (Allan C. et al., 1993, Biochem J, 290, 449-455). This fragment corresponds to amino acid sequence 173-267 of the full length ApoA-1 protein. Processing in ApoA-1 occurs at position R184. The size predicted for the C-terminal fragment based on this cleavage is 9.3 kDa.

Serum obtained from healthy males was pooled and depleted using the Multiple Affinity Removal System (MARS) level I (Agilent) removing the 6 most abundant proteins (albumin, alfa-1-antitrypsin, haptoglobin, IgG, IgA, transferrin). 3× Loading buffer (125 mM Tris-HCl/4% SDS/50% glycerol/0.02% Bromophenol Blue/10% beta mercaptoethanol) was added to the depleted serum (at 250 μg/ml) after a 2 fold concentration of the sample. Approximately 10 μg was loaded on the SDS-PAGE gel. A 4-20% gradient gel (Precise gel, Pierce) was used to separate the protein sample. The Kaleidoscope prestained marker (Biorad) was loaded for molecular weight reference. After separation, the protein material was transferred to Immobilon-P^{SQ} membranes (Millipore) by electroblotting. The membrane was blocked with blocking buffer (5% BSA in TBS) followed by probing with the primary antibody AI-4.1 directed against the C-terminus of ApoA-1. To this end, a 1/15 dilution of crude mouse hybridoma supernatant in incubation buffer (0.5% BSA in TBS+0.1% Tween20) was used. After washing, secondary antibody was added at a 1/6000 dilution in incubation buffer. HRP activity was revealed by using the West pico chemiluminescence substrate (Pierce) and Hyperfilm™ ECL films (Amersham) using a Hypercassette™ (Amersham) in combination with an automated developer (Fujifilm S/N244-FPR-001).

The western blot confirms a processing event of the expected size in the depleted serum of male donors. As the antibody was specifically raised against the C-terminal part starting from amino acid 173, and the size of the observed fragment is close to the size of this C-terminal part, it can be expected that the observed ApoA-1 starts very close to position 173, which fits with the observation of a processing event after R184 in the COFRADIC analysis of example 1.

### Example 3

### Analysis of the Stability of the ApoA1-R184P-Mutant and Wild-Type ApoA1

Wild-type (WT) ApoA1 and ApoA1 variants with mutated cleavage sites are produced recombinantly in bacteria to monitor stability of the protein in blood. Different mutants are generated by mutagenesis of the expression construct. We opted for a R184-P mutant, as this mutation naturally occurs in some carriers, and for the R184-H mutant which is expected to have less effect on the secondary structure of the protein. Other mutations surrounding the R184 site as exemplified above are introduced as well.

### Protocol:

The cDNA of human ApoA1 in the pENTR™221 vector (clone ID: IOH7318, Gateway® system, Invitrogen) can be used as template DNA for a site directed mutagenesis reaction. Mutagenic primers comprising the sequence for the mutated amino acid are used in the PCR reaction of the Quickchange™ mutagenesis kit. An additional restriction site alteration is introduced by the mutagenic primers to facilitate detection of mutated sequences. After verification of mutated sequences by sequencing, the cDNA sequence is transferred to the pET11d expression vector (Novagen) by recombination-assisted cloning, direct transfer with compatible restriction enzymes, or by PCR-based cloning. The signal sequence is omitted in the final expression construct and is replaced by a N-terminal HIS-tag to empower purification by nickel affinity chromatography. The HIS-tag is followed by a TEV protease cleavage site. Inducible expression is obtained with the BL21 bacterial system. A number of mutated constructs were created that have mutations at and around the R184 site. After expression, the proteins are purified by nickel affinity chromatography. Further purification is obtained by gel filtration with optimal resolution in the Mr 30000 range.

The protein sample can then be used for spiking in blood or serum. Different quantities of the recombinant proteins (8-40-200-1000 μg/ml) are diluted in serum. Samples are collected at different time points (t0, 30′, 1 hr, 2 hr, 4 hr, 8 hr, 16 hr) and used for Western analysis. For these experiments the HIS-tag is not removed by TEV protease activity. Stability is monitored by Western blot directed against the HIS-tag. Mouse monoclonal anti-HIS-tag antibody is provided by R&D systems (MAB050) and is used at 1 μg/ml in TBS-T/milk solution. Secondary HRP-coupled donkey anti-mouse antibody is from Abcam (ab7061) and is used for revelation at 0.5 μg/ml in TBS-T/Milk.

### Results:

Western analysis is expected to reveal prolonged half-lives for ApoA1 proteins carrying mutations in the R184 site. Sensitivity to proteolytic activity is expected to be reduced in these mutants.

### Example 4

### Stability of ApoA1 Variants in Mice

For further evaluation of ApoA1 stability, in vivo experimentation in mice is employed. Recombinant proteins are administered to mice deficient in ApoAI, and the stability in blood is monitored by Western analysis. The difference in HDL levels after administering the different ApoA1 variants is evaluated by FPLC separation of mouse plasma followed by quantification of cholesterol, phospholipids and triglycerides in the different fractions, where higher fractions correspond to HDL particles.

### Protocol:

Apol deficient mice (B6.129P2-Apoa1^{tm1Unc}/J, Jackson Labs) are injected intravenously with the recombinant protein preparations described higher. Injections of recombinant ApoA1 proteins to obtain a final concentration of 40, 200 and 1000 mg/ml in blood will be used to better visualize increased stability.

Stability of the proteins in serum is monitored by using anti-HIS antibody and Western blot. In addition, the HIS tag is removed from the recombinant proteins by TEV protease treatment. After intravenous injection of the processed and purified proteins, stability is monitored by Western blotting using the procedure and the antibodies described in part 1.

Fast protein liquid chromatography (FPLC) is used to fractionate mouse plasma. 20 μl of mouse plasma is fractionated on a Sepharose 6 PC column (GE Healthcare) and eluted with PBS. 25 fractions of 50 μl volume each are collected. Levels of total cholesterol and phospholipids in the fractions are determined using the Cholesterol CII and the Phospholipids B kit respectively (both from Wako Chemicals USA, Inc.). Triglyceride content in the fractions is monitored by INFINITY triglycerides (Thermo DMA). Total ApoA1 levels are determined using Autokit ApoA1 (Wako Diagnostics USA, Inc), and are verified by Western blot using the protocol and the antibodies described higher. Higher FPLC fractions correspond to the HDL particles (fractions 14-20) while VLDL and LDL can be found in the lower FPLC fractions (respectively in fractions 1-9 and fractions 10-13).

### Results:

The concentration of total cholesterol, phospholipids and ApoA1 is expected to be significantly larger in the HDL fractions for the mutant proteins when compared to the wild type protein, suggesting an accumulation of functional mutant ApoA1 in these particles. An increased formation of these HDL particles suggests increased reverse cholesterol transport and implies an improved efficacy of recombinant ApoA1 R184 mutants when used for treatment of cardiovascular disease.

### Example 5

### Linkage of the ApoA1 R184→P Genetic Variation to Increased HDL Levels in Carriers

A previous study shows the occurrence of a natural genetic variation (SNP) resulting in a R184→P alteration (NCBI SNP ID: rs5078). The study was initiated to evaluate genetic variation in a list of candidate genes linked to blood pressure homeostasis.

### Protocol:

A large study population of people with high levels of HDL (in upper 2.5 percentile of population) and people with normal levels of HDL will be used for sequencing of the affected region. Primers were designed to amplify the genomic region containing the genetic variation. The occurrence of the R184→P variation is then counted in the complete study population and tested for statistically significant linkage to high HDL levels.

### Results:

Linkage of the R184→P genetic variation to increased HDL levels is to be expected, supporting increased stability of this ApoA1 variant, resulting in increased HDL levels.

1. A method for increasing the half-life and/or modulating the activity of one or more protein(s) comprising 1) identifying the novel internal proteolytic cleavage site(s) in said one or more protein(s) using N-terminal or C-terminal technology, 2) modifying said identified proteolytic cleavage site(s) in said one or more protein(s) such that the sensitivity of said one or more protein(s) towards proteolytic cleavage at said identified site(s) is modulated.

2. The method of claim 1, wherein, sensitivity of at least one of said one or more protein(s) towards proteolytic cleavage at said site(s) is altered, thereby altering its stability of the at least one protein(s) in an in vivo production process comprising a transgenic animal or a microbacterial system.

3. A method of detecting naturally occurring SNPs that are connected to a disease or disorder related to proteolytic cleavage of a protein comprising the steps of:
a) identifying the proteolytic cleavage site(s) that lead to protein cleavage during the production process in said protein using N-terminal or C-terminal technology,
b) searching an SNP database for mutations in the isolated protein that correspond to the newly identified proteolytic cleavage site in step a).

4. A method for diagnosing a disease or disorder related to proteolytic cleavage of a protein comprising the steps of detecting one or more SNPs identified by the method of claim 3, in a sample of said patient.

5. The method of claim 1, wherein said one or more protein(s) is or forms part of a protein-based medicament, a pharmaceutical composition, a vaccine, or a diagnostic composition.

6. The method of claim 1, wherein the one or more protein(s) is produced synthetically or recombinantly.

7. The method of claim 1, wherein the modification of the identified proteolytic cleavage site comprises introducing one or more point mutation(s) in the nucleic acid coding sequence of the protein(s) at a position overlapping with and/or surrounding said identified proteolytic cleavage site(s), thereby altering the amino acid sequence of the protein(s) and subsequently blocking, inhibiting or reducing proteolytic cleavage.

8. The method of claim 1, wherein the modification of the identified proteolytic cleavage site(s) comprises chemical modification of one or more side chains of the amino acid residues overlapping with and/or surrounding said identified proteolytic cleavage site(s), subsequently blocking, inhibiting or reducing proteolytic cleavage.

9. The method of claim 1, wherein the modification of the identified proteolytic cleavage site(s) comprises introducing one or more non-natural amino acids encoded by specific non-natural codons which are introduced in the coding sequence of the target protein(s).

10. The method of any of claim 1, wherein the modulation of the activity of one or more protein(s) comprises binding an affinity ligand or a binding molecule to the proteolytic cleavage site(s) in the one or more protein(s) or to its one or more protease(s) of the one or more protein(s), thereby preventing or reducing the interaction between the protein(s) and the protease(s).

11. The method of claim 1, wherein the proteolytic cleavage at the identified proteolytic cleavage site(s) is reduced by inhibiting the protease(s) responsible for cleaving the protein(s) of interest which comprises adding one or more inhibiting agent(s) to a mixture comprising the protein(s).

12. The method of claim 1, wherein identification of the proteolytic cleavage site(s) in one or more protein(s) present in a protein mixture comprises the steps of:
a) optionally selecting a protein of interest from the protein mixture using a specific binding molecule or a combination of several specific binding molecules,
b) modifying or labelling all true and/or novel internal N-termini or C-termini of the protein(s) in the protein mixture,
c) cleaving or hydrolysing the proteins in the protein mixture into peptides with e.g. trypsin, chymotrypsin and the like,
d) optionally separating the modified or labelled N-terminal or respective C-terminal peptides from the non-modified or non-labelled peptides in the protein mixture,
e) analyzing only the modified or labelled N-terminal or respective C-terminal peptides from the mixture using mass-spectrometric methods, and
f) identifying all internal proteolytic cleavage site(s) of said one or more protein(s) in said protein mixture.

13. The method of claim 12, wherein the N-terminal or C-terminal modification step b) is done by blocking the true and/or novel internal N-termini or respective C-termini with a specific agent and the optional separation step d) is done by using aminopeptidase or respective carboxypeptidase degrading only the non-protected peptides in the protein mixture into single amino acid residues.

14. The method of claim 12, wherein the N-terminal or C-terminal labelling step b) is done by addition of a capturing-molecule on the true and/or novel internal N-termini or respective C-termini of the protein(s) and wherein the optional separation step d) is done by capturing only the labelled peptides on a solid support or by capturing only the non-labelled peptides on a solid support.

15. The method of claim 14, wherein said capturing-molecule is selected from the group of beads, glass beads, controlled-pore silicate glass beads such as biotin, PITC or DITC, an organic cyclic compound such as a crown ether or a derivative thereof, MIPs, DARPins, a fluorous or cis-diol moiety or any other molecule designed to selectively bind to the primary amine groups of the N-termini or any other molecule that binds selectively to the novel C-termini of proteins, and wherein the separation step is done by column purification, affinity capture, filtration, centrifugation, magnetic capture, matrix capturing or the like.

16. The method of claim 12, wherein the protein mixture is derived from a complex body sample selected from the group of blood, plasma, serum, urine, faeces, saliva, cerebrospinal fluid, nipple aspirate, ductal lavage, sweat or perspiration, tumor exudates, joint fluid (e.g. synovial fluid), inflammation fluid, tears, semen, vaginal secretions and tissue biopsies and wherein the protein mixture comprises one or more proteins, wherein said proteins can be present one or more isoforms.

17. The method of claim 12, wherein the analysis of the N-terminal or C-terminal peptides is done by using electrospray ionization mass spectrometry, ion trap mass
spectrometry, hybrid ion trap mass spectrometry coupled to quadrupole, time-of-flight mass spectrometry, or a reversed phase-high performance liquid chromatography system connected to a nanospray ionization hybrid ion trap-fourier transform mass spectrometer.
